(19) United States
(12) Patent Application Publication
(10) Pub. No.: US 2004/0101873 A1
(43) Pub. Date: May 27, 2004

(54) Method and apparatus for validating DNA sequences without sequencing

(76) Inventor: Gregory T. Went, Mill Valley, CA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/403,751

(22) Filed: Mar. 31, 2003

Related U.S. Application Data
(63) Continuation-in-part of application No. 10/360,003, filed on Feb. 6, 2003, now abandoned.
(60) Provisional application No. 60/354,640, filed on Feb. 6, 2002.

(51) Int. Cl. C12Q 1/68; G06F 19/00; G01N 33/48; G01N 33/50

(52) U.S. Cl. 435/6; 702/20

(57) ABSTRACT

The present invention provides a system comprising methods by which the sequence of a biologically or non-biologically derived nucleic acid can be determined without sequencing. The methods preferably compare the molecular masses of subsequences generated from the target sequence with predicted molecular masses by a database look-up step. Computer-implemented methods are provided to analyze the experimental results and to determine any sub-regions of the nucleic acid containing one or more variations.

### REFERENCE TO PRIOR APPLICATION

This application is a continuation-in-part application of U.S. Ser. No. 10/360,003, filed Feb. 6, 2003, which claims the benefit of U.S. Provisional Application No. 60/354,640, filed Feb. 6, 2002, the contents of which are hereby incorporated by reference into the present specification in their entireties.

### FIELD OF THE INVENTION

The field of this invention is nucleic acid molecule sequence classification, identification or determination; more particularly it is the validation of large fragments of nucleic acid or genes in a sample without performing de novo sequencing, as well as methods for screening nucleic acids for polymorphisms or mutations by analyzing fragmented nucleic acids using mass spectrometry.

### BACKGROUND OF THE INVENTION

The sequence of the human genome contains approximately 3×10⁹ nucleotides, essentially all of which is publicly available as a result of the Human Genome Project. However, this is a consensus sequence derived for the genomic sequence from relatively few individuals, and the heterogeneity and complexity of both sequence polymorphisms and the splicing pattern of the human genome has been heretofore inadequately explored and characterized.

With this draft in hand of the primary DNA sequence of the human genome, one of the next large undertakings in biology is the assembly of a complete set of full-length cDNAs and their variants for all of the 30,000 or so genes. This is an essential step in understanding the function of all genes as well as a starting point for the development of the next generation of biotherapeutics and target-specific small molecule drugs. While the existing sequence information derived from the human genome project and the EST sequencing projects enables accurate predictions to be made of the primary sequence of many full-length cDNAs, the assembled cDNAs still must be isolated and sequence validated to determine subtle genetic alterations, e.g. point mutations, genetic polymorphisms, or splicing variants, that may not be readily discerned by common, high-throughput laboratory methods such as gel electrophoresis.

Thus, a method that is able to sequence validate DNA and DNA clones representing all the polymorphisms, splice variants, mutations, and any other causes of heterogeneity of the human genome is useful. Such a method would also provide an economically desirable means for determining novel secreted protein drugs, antibody and small molecule targets, and reagents for large scale functional studies in an economically viable way.

Strategies directed towards studying novel gene function involve isolating full length cDNAs and then cloning these cDNAs into expression vectors. A current impediment is the validation process—confirming that the cDNA sequence inserted into the vector is an intact, in frame, exact representation of the wild type sequence. Conventional DNA sequencing requires the redundant sequencing of several, overlapping clones of 400 bp length to properly confirm sequence identity, exon ordering and the degree of error introduced into the sequence. While Sanger sequencing of partial or full-length cDNAs will detect any variations at the molecular level, this strategy is prohibitively expensive and an unnecessary tact given that most of the sequence for each cDNA in question will be invariant from that predicted based on the relevant reference cDNA sequence. Sequencing by hybridization has been proposed (See, e.g., U.S. Pat. Nos. 6,451,996, 5,667,972, 6,018,041, 5,510,270, 5,871,928, and 6,300,063), but is inefficient at determining exon order and inadequate in resolving power. More recently, mass spectrometry has been used to sequence nucleic acids (See, e.g., U.S. Pat. Nos. 6,268,131 and 6,140,053) and to identify mutations in nucleic acids (See, e.g., U.S. Pat. Nos. 6,051,378 and 6,500,621) but none of these methods are cost effective at validating large numbers of these larger DNA fragments. Any improved method for sequence validation will apply to other genomes as well. For all of the above purposes, a rapid, low cost means of validating large fragments of DNA would have a major impact on nucleic acids research and diagnostics. The general availability of wild type sequence for the mammalian and pathogen genomes of interest creates a new application, namely sequence validation.

Genetic polymorphisms such as mutations can manifest themselves in several forms, such as point mutations, wherein a single base is changed to one of the three other bases, deletions, wherein one or more bases are removed from a nucleic acid sequence and the bases flanking the deleted sequence are directly linked to each other, and insertions, wherein new bases are inserted at a particular point in a nucleic acid sequence adding additional length to the overall sequence. Large insertions and deletions, often the result of chromosomal recombination and rearrangement events, can lead to partial or complete loss of a gene. Of these forms of mutation, in general the most difficult type of mutation to screen for and detect is the point mutation, because it represents the smallest degree of molecular change. Detection of all of the polymorphisms associated with a single gene, whether at the genomic level or simply for the entire pools of exons that comprise that gene, remains impractical in research or diagnostic applications owing to the high cost of sub-cloning and Sanger sequencing.

Thus, it is an object of this invention to provide a method for rapidly identifying regions of a nucleic acid sequence that vary from wild-type. It is a further object of this invention to provide a method to determine polymorphisms in nucleic acid sequences by focusing only on the region of polymorphism. In nearly all practical cases, the rate of polymorphism per base pairs is between approximately 1 every 10,000 and 1 every 100 in the extreme. Other objects of the invention will be readily apparent to those of ordinary skill in the art from the description of the invention in the specification. As explained in detail herein, the methods of the invention separate (via fragmentation, for example) the nucleic acid molecule sample into overlapping fragments and independently validate the molecular weight of each fragment and their corresponding plus and minus strands. Owing to the extreme low probability of compensating variants, an exact match to the wild type sequence can be readily assumed to be invariant. Only those small number of fragments harboring variant masses need be sequenced in detail, drastically reducing the time and cost of sequence validation. The present invention, therefore, allows for the rapid validation of sequence of a nucleic acid molecule, and concomitant determination of any sequence polymorphisms, without the need to sequence the portion of nucleic acids that do not vary from the wild type sequence.

### SUMMARY OF THE INVENTION

The present invention provides a method for validating the sequence of a nucleic acid or detecting polymorphisms within a nucleic acid without sequencing the entirety of the nucleic acid.

One aspect the present invention provides methods of validating the sequence of a test double stranded nucleic acid, by contacting the test double stranded nucleic acid with one or more separation means, such that two or more double stranded nucleic acid fragments are generated from said test nucleic acid; generating one or more output signals from each of the fragments, the output signals including a representation of the molecular mass of each of the fragments; and comparing the one or more output signals with a set of output signals known or predicted to be produced by a nucleic acid of identical sequence to the test nucleic acid, whereby the sequence of the test nucleic acid is validated. In an embodiment of the invention the separation means is a recognition means. In the practice of the invention, each recognition means recognizes a different target nucleotide subsequence or a different set of target nucleotide subsequences of the test nucleic acid. In a related embodiment of the invention, the test nucleic acid is contacted with one or more recognition means that are restriction enzymes, such as restriction endonucleases. In another embodiment, the output signals are derived from mass spectrometry. Methods of mass spectrometry of the present invention include, but are not limited to, ion cyclotron resonance mass spectrometry, electrospray ionization fourier transform ion cyclotron resonance mass spectrometry, matrix-assisted laser desorption ionization mass spectrometry, quadropole ion trap mass spectrometry, magnetic/electric sector mass spectrometry and time-of-flight mass spectrometry. An optional aspect of the invention is the inclusion of internal calibrants or internal self-calibrants in the set of nonrandom length fragments to be analyzed by mass spectrometry to provide improved mass accuracy. In embodiments of the invention the target double stranded nucleic acid is DNA or double stranded RNA. Sources of DNA include genomic DNA, cDNA, and DNA generated by polymerase chain reaction (PCR).

In embodiments of the invention, the method may be repeated one, two, three or more times, under conditions such that the size of each of the two or more nucleic acid fragments is decreased with each repetition. In embodiments of the invention, the two or more double stranded nucleic acid fragments generated are each under a certain length, e.g., under 500 bases, 200 bases, 100 bases, 50 bases, or 20 bases in length.

Another aspect of the invention provides a method for identifying all or substantially all of the DNA fragments encoding polymorphisms in a test double stranded nucleic acid, the method including contacting the test double stranded nucleic acid with one or more separation means, such that two or more double stranded nucleic acid fragments are generated from the test nucleic acid; generating one or more output signals from each of the fragments, the output signal including a representation of the molecular mass of each of the fragments; and comparing the one or more output signals with a set of output signals of a reference nucleic acid of identical sequence, whereby a difference in the one or more output signals of one or more nucleic acid fragments indicates a difference in the sequence of the one or more nucleic acid fragments, thereby identifying all or substantially all of the DNA fragments encoding polymorphisms in the test nucleic acid.

In an embodiment of the invention, the method further includes identifying the one or more nucleic acid fragments having the polymorphism; and repeating the method one or more times, under conditions such that the size of each of the two or more nucleic acid fragments is decreased with each repetition. In a related embodiment the method further includes sequencing the nucleic acid fragments with output signals different from the output signals of the reference nucleic acid.

In another aspect, the invention provides a method for detecting a polymorphism in a target nucleic acid, the method including obtaining from the target nucleic acid a population of nucleic acid fragments in double stranded form, wherein the population essentially comprises the entirety of fragments generated from non-randomly fragmenting a double-stranded target nucleic acid, and determining the molecular masses of each of the double-stranded nucleic acid fragments of the population. In an embodiment of the invention, the method further includes comparing the molecular mass of each of the double-stranded nucleic acid fragments with the molecular masses known or predicted to be produced by a double stranded reference nucleic acid; and sequencing the nucleic acid fragments with molecular masses different from the molecular masses of the reference nucleic acid.

Another aspect of the invention provides a method for detecting a variation in a nucleic acid sequence among two individuals, the method including independently contacting a first nucleic acid from a first individual and a second nucleic acid from a second individual with one or more separation means, such that two or more double stranded nucleic acid fragments are generated from each of the first nucleic acid and the second nucleic acid; generating one or more output signals from each of the fragments, the output signal including a representation of the molecular mass of each of the fragments; and comparing the one or more output signals generated from the first nucleic acid with the one or more output signals generated from the second nucleic acid, whereby a variation in a nucleic acid sequence among two individuals is detected.

Another aspect of the invention provides a method for determining paternity of an offspring, the method including independently contacting a first nucleic acid from a first individual and a second nucleic acid from a second individual with one or more separation means, such that two or more double stranded nucleic acid fragments are generated from each of the first nucleic acid and the second nucleic acid; generating one or more output signals from each of the fragments, the output signal including a representation of the molecular mass of each of the fragments; and comparing the one or more output signals generated from the first nucleic acid with the one or more output signals generated from the second nucleic acid, thereby determining the paternity of the first individual relative to the second individual.

A further aspect of the invention includes a method for identifying a polymorphism in a target double stranded nucleic acid, the method including the steps of contacting the target double stranded nucleic acid with one or more restriction enzymes, such that two or more double stranded nucleic acid fragments are generated from the target nucleic acid; determining the molecular masses of each of the double-stranded nucleic acid fragments; comparing the molecular masses of each of the double-stranded nucleic acid fragments with the molecular masses of the double-stranded nucleic acid fragments known or predicted to be produced by a double stranded reference nucleic acid of identical sequence to the target nucleic acid; repeating these steps one or more times, under conditions such that the size of each of the two or more nucleic acid fragments is decreased with each repetition; and sequencing the nucleic acid fragment(s) with molecular masses different from the molecular masses of the double-stranded nucleic acid fragments of the reference nucleic acid.

An other aspect of this invention is a processor for analyzing nucleic acid sequences comprising a selecting module that enables a user to select one or more textual strings corresponding to one or more genes; in response to the user's selection, a providing module that provides a first set of nucleic acid sequence fragments comprising the fragments predicted to be generated by contacting a first double stranded nucleic acid molecule with at least one separation means, said first set of nucleic acid sequence fragments associated with the selected one or more textual stings; an evaluating module that evaluates each of the first set of nucleic acid sequence fragments to predict the mass of each fragment of the first set of nucleic acid sequence fragments; a retrieving module that retrieves experimental results comprising the mass of each of a second set of nucleic acid sequence fragments, said second set of nucleic acid sequence fragments generated by contacting a second double stranded nucleic acid molecule with said at least one separation means; a validating module that validates each of the first set of nucleic acid sequence fragments by evaluating the mass of each fragment of the first set of nucleic acid sequence fragments against the mass of each fragment of the second set of nucleic acid sequence fragments.

In the practice of this aspect of the invention the processor may further comprise a storing module that stores the results of the validation. As part of this aspect of the invention, the separation means can be a recognition means, such as a restriction endonuclease, preferably a type 2 restriction endonuclease. The process for evaluating the mass of each fragment preferably comprises performing mass spectrometry on each fragments. Applicable means of mass spectrometry can include ion cyclotron resonance mass spectrometry, electrospray ionization fourier transform ion cyclotron resonance mass spectrometry, matrix-assisted laser desorption ionization mass spectrometry, quadropole ion trap mass spectrometry, magnetic/electric sector mass spectrometry and time-of-flight mass spectrometry.

In a preferred embodiment of this aspect of the invention the nucleic acid is DNA, however it can alternatively be nucleic acid is double stranded RNA.

A further aspect of this invention includes a method for analyzing nucleic acid sequences comprising enabling a user to select one or more textual strings corresponding to one or more genes; in response to the user's selection, providing a first set of nucleic acid sequence fragments associated with the selected one or more textual strings, said first set of nucleic acid sequence fragments comprising the fragments predicted to be generated by contacting a first double stranded nucleic acid molecule with at least one separation means; evaluating each of the first set of nucleic acid sequence fragments to predict the mass of each of the first set of nucleic acid sequence fragments; retrieving experimental results comprising the mass of each of a second set of nucleic acid sequence fragments, said second set of nucleic acid sequence fragments generated by contacting a second double stranded nucleic acid molecule with said at least one separation means; and validating the each of the first set of nucleic acid sequence fragments by evaluating the mass of the each of the first set of nucleic acid sequence fragments against the mass of each of the second set of nucleic acid sequence fragments.

In the practice of this aspect of the invention the method may further comprise a step of storing the results of the validation. As part of this aspect of the invention, the separation means can be a recognition means, such as a restriction endonuclease, preferably a type 2 restriction endonuclease. The process for evaluating the mass of each fragment preferably comprises performing mass spectrometry on each fragments. Applicable means of mass spectrometry can include ion cyclotron resonance mass spectrometry, electrospray ionization fourier transform ion cyclotron resonance mass spectrometry, matrix-assisted laser desorption ionization mass spectrometry, quadropole ion trap mass spectrometry, magnetic/electric sector mass spectrometry and time-of-flight mass spectrometry.

In a preferred embodiment of this aspect of the invention the nucleic acid is DNA, however it can alternatively be nucleic acid is double stranded RNA.

Another aspect of this invention provides a processor for analyzing nucleic acid sequences comprising selecting means that enables a user to select one or more textual strings corresponding to one more genes; in response to the user's selection, providing means that provides the mass of each fragment of a first set of nucleic acid sequence fragments associated with the selected one or more textual strings; evaluating means that evaluates each of the first set of nucleic acid sequence fragments to predict the mass of each fragment of the first set of nucleic acid sequence fragments for at least one separation means; retrieving means that retrieves experimental results comprising the mass of each fragments in a second set of nucleic acid sequence fragments for said at least one separation means; validating means that validates the first set of nucleic acid sequence fragments by evaluating the mass of each fragment of the first set of nucleic acid sequence fragments against the experimental results of the mass of each fragment of the second set of nucleic acid sequence fragments; and storing means that stores the results of the validation.

A further aspect of this invention provides a processor readable medium for analyzing nucleic acid sequences, said medium comprising a first processor readable program code for enabling a user to select one or more textual strings corresponding to one or more genes; in response to the user's selection, a second processor readable program code for providing a first set of nucleic acid sequence fragments associated with the selected one or more textual strings; a third processor readable program code for evaluating each of the first set of nucleic acid sequence fragments to calculate the mass of each fragment of the first set of nucleic acid sequence fragments, said first set of nucleic acid sequence fragments comprising the fragments predicted to be generated by contacting a first double stranded nucleic acid molecule with at least one separation means; a fourth processor readable program code for retrieving experimental results of the determination of the mass of each fragment of a second set of nucleic acid sequence fragments, said second set of nucleic acid sequence fragments comprising the fragments generated by contacting a second double stranded nucleic acid molecule with said at least one separation means; a fifth processor readable program code for validating the sequence of the first nucleic acid molecule by evaluating the mass of each fragment of the first set of nucleic acid sequence fragments against the experimental results of the mass of each of the second set of nucleic acid sequence fragments; and a sixth processor readable program code for storing the results of the validation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1a depicts the nucleic acid sequence of a Pan1 nucleic acid (SEQ ID NO: 1) isolated from hamster. FIG. 1b depicts the nucleic acid sequence of Pan2 (SEQ ID NO: 2) isolated from hamster.

FIG. 2 demonstrates the pair wise sequence alignment of Pan1 and Pan2 nucleic acids.

FIG. 3 indicates the predicted AciI and HaeIII restriction enzyme sites within Pan1 and Pan2 cDNAs. The hatched boxes below the genes indicate regions of sequence divergence between Pan1 and Pan2 sequences.

FIG. 4 is a schematic representation of an embodiment of the sequence validation method of the present invention using a Pan1 cDNA amplicon.

FIG. 5a is a partial ESI-FTICR-MS spectra (M/Z of 952.5-957.5) of RE fragments derived from a Pan1-like cDNAs; FIG. 5b is the deconvolution and analysis of the same partial ESI-FTICR-MS Spectra of RE fragments derived from a Pan1-like cDNAs.

FIG. 6a is a partial ESI-FTICR-MS spectra (M/Z of 1017.5-1027.0) of RE fragments derived from a Pan1-like cDNAs; FIG. 6b is the deconvolution and analysis of the same partial ESI-FTICR-MS Spectra of RE fragments derived from a Pan1-like cDNAs.

FIG. 7 is a schematic representation of an embodiment of the polymorphism scanning method of the present invention using genomic DNA (gDNA).

FIG. 8 is a schematic representation of an embodiment of the polymorphism scanning method of the present invention using the CFTR exon and intron junction regions.

FIG. 9 depicts an embodiment of the invention where multiple separation means, in this instance restriction endonuclease digestion, of double stranded DNA yields complete coverage of the sequence of the Pan1 gene overcoming any lower limits of resolution in current mass spectrometry methods. In the figure, lightly shaded fragment regions of the gene will be observed, whereas darker shaded fragment regions will be missed. In order to ensure complete coverage of the entire sequence of the nucleic acid, multiple restriction endonucleases are employed and samples are run in tandem.

FIG. 10 depicts a flow diagram demonstrating an embodiment of the clone validation system of the invention.

FIG. 11 depicts a flow diagram demonstrating an embodiment of the method of building a nucleic acid reference database, in this instance a method of building a cDNA reference database.

FIG. 12 depicts a flow diagram demonstrating an embodiment of the method for predicting fragments of cleaved nucleic acid molecules, in this instance a method of predicting restriction enzyme-cleaved fragments of a cDNA sample.

FIG. 13 depicts a flow diagram demonstrating an embodiment of the method of generating nucleic acid fragments from clones by contacting nucleic acid molecules with separation means, in this instance contacting clones containing the nucleic acid molecules with restriction enzymes.

FIG. 14 depicts a flow diagram demonstrating an embodiment of the method of generating fragment data for comparison of predicted and experimentally derived fragment sets.

FIG. 15 depicts a flow diagram demonstrating an embodiment of the method of comparing the predicted and experimentally derived fragment sets.

FIG. 16 depicts a flow diagram describing an embodiment of the clone validation system of the invention.

FIG. 17 depicts a flow diagram describing a second embodiment of the clone validation system of the invention.

### DETAILED DESCRIPTION OF THE INVENTION

The present invention is directed in part to methods of validating the entire sequence of nucleic acids and for localizing polymorphisms in nucleic acid sequences derived from PCR, expression cloning, genomic cloning and the like using mass spectrometry. The methods described herein can be performed iteratively in order to confirm the sequence of the nucleic acid without sequencing the nucleic acid or, alternatively, to provide detailed information about the nature and location of polymorphisms in the target nucleic acid. The method and apparatus is especially useful for the analysis and validation of fragments ranging from approximately 1 kb up to approximately 100 kb, but may be adapted for even higher weight fragments.

The present invention involves obtaining from a target nucleic acid, using a variety of nonrandom fragmentation techniques, a set of two or more double stranded nucleic acid fragments and comparing the set of fragments with a set of fragments known or predicted to be produced by a double stranded reference nucleic acid of identical sequence to the predicted sequence of the target nucleic acid. The reference nucleic acid may be, e.g., the wild type nucleic acid or may be a nucleic acid having a consensus sequence, i.e., a composite sequence generated by averaging two or more nucleic acid sequences. Most wild type sequences for the genes and genomes of interest are known and are stored in databases. Wild type refers to a standard or reference nucleotide sequence to which variations are compared. As defined, any variation from wild type is considered a mutation, including naturally occurring sequence polymorphisms, insertions, deletions, substitutions, and inversions. The term mutation encompasses all the above-listed types of differences from wild type nucleic acid sequence.

The target nucleic acid can be single-stranded or double-stranded DNA, RNA or hybrids thereof, from any source, preferably from a mammalian source, e.g., a human, although any source from which one is capable of isolating nucleic acids can be used in the methods described herein, including pathogens and viruses. Uncommon DNA structures including triple stranded and quadruple stranded DNA are also included in the present invention. The target nucleic acid of the present invention can also be synthesized by methods known to those skilled in the art. When the target nucleic acid is RNA, the RNA is preferably made double-stranded. If desired, the target nucleic acid can be an RNA/DNA hybrid, wherein either strand can be designated the plus or forward (+) strand and the other, the minus or reverse (−) strand. The target nucleic acid is generally a nucleic acid which must be screened to determine all or substantially all of the polymorphisms, such as mutations. The corresponding target nucleic acid derived from a wild type source is referred to as a reference nucleic acid. The target nucleic acids can be obtained from a source sample containing nucleic acids and can be produced from the nucleic acid by PCR amplification or other amplification technique. The target nucleic acids can be of any size capable of being fragmented by a separation means, e.g., a restriction enzyme.

Nonrandom length fragments are nucleic acid molecules generated by nonrandom fragmentation of a target nucleic acid molecule by any separation means, such that two or more double stranded nucleic acid fragments are generated. In the practice of the methods of this invention, nonrandom length fragment set(s) generated from the target nucleic acid molecule is(are) compared against reference fragment set(s) prepared from a predicted fragmentation of a reference nucleic acid molecule to validate the sequence of the target nucleic acid molecule. The preferred method of comparing the nonrandom length fragment set(s) to the reference fragment set(s) is to determine the masses of sets of nonrandom length fragments, and to determine the mass of essentially every fragment resulting from the fragmentation of the target double stranded nucleic acid. Thus, the methods described herein preferably use mass spectrometry to determine the masses of the set or sets of nonrandom length fragments and compare the output of mass spectrometry to the predicted output of the reference fragment set. The resolving power of the mass spectral analyses of the present invention allow the detection of a very small mass change (on the order of 0.4 Da or smaller) in a nonrandom length fragment, while the mass change of a single base substitution is at least 9 Da (representing a change from A to T).

The methods described herein do not require sequencing of the target nucleic acid in order to confirm that the target nucleic acid has the identical sequence of the reference nucleic acid, or alternatively, to identify the nature and presence of all or substantially all of the mutations within the target nucleic acid. Instead, the methods of the present invention allow the comparison of the individual masses of a set of nucleic acid fragments derived from a target nucleic acid with masses of nucleic acid fragments known or predicted to be produced by a double stranded reference nucleic acid of identical sequence to the predicted sequence of the target nucleic acid. By identifying a nucleic acid fragment from the target nucleic acid whose mass differs from the masses of the reference nucleic acid fragments, a nucleic acid fragment containing a polymorphism can be detected. The methods of the present invention can be performed iteratively, such that the size of the nucleic acid fragment containing a polymorphism is successively reduced with each repetition. The specific nature and location of the polymorphism can then be identified by conventional sequencing methods, e.g., Sanger sequencing using dideoxy termination and denaturing gel electrophoresis (Sanger, F., Nichlen, S. & Coulson, A. R. Proc. Natl. Acad. Sci. USA 75, 5463-5467 (1977), Maxam-Gilbert sequencing using chemical cleavage and denaturing gel electrophoresis (Maxam, A. M. & Gilbert, W. Proc. Natl. Acad. Sci. USA 74, 560-564 (1977)), pyro-sequencing detection of pyrophosphate (PPi) released during the DNA polymerase reaction (Ronaghi, M., Uhlen, M. & Nyren, P. Science 281, 363, 365 (1998)), and sequencing by hybridization (SBH) using oligonucleotides (Lysov, I., Florent'ev, V. L., Khorlin, A. A., Khrapko, K. R. & Shik, V. V. Dokl Akad Nauk SSSR 303, 1508-1511 (1988); Bains W. & Smith G. C. J Theor Biol 135, 303-307(1988); Drnanac, R., Labat, I., Brukner, I. & Crkvenjakov, R. Genomics 4, 114-128 (1989); Khrapko, K. R., Lysov, Y., Khorlyn, A. A., Shick, V. V., Florentiev, V. L. & Mirzabekov, A. D. FEBS Lett 256. 118-122 (1989); Pevzner P. A. J Biomol Struct Dyn 7, 63-73 (1989); Southern, E. M., Maskos, U. & Elder, J. K. Genomics 13, 1008-1017 (1992)).

The nonrandom fragmentation techniques of the invention are any methods of fragmenting nucleic acids that provide a defined set of nonrandom length fragments, where that set of nonrandom length fragments may be reproducibly obtained by using the same nonrandom fragmentation method on the same target nucleic acid or its wild type version. The methods used for nonrandom fragmentation are designed to optimize the ease of analyzing the resulting fragment set mass spectral data, e.g., by obtaining a range of fragment sizes that avoids significant overlap of mass peaks. The nonrandom fragmentation techniques of the invention include enzymatic nonrandom fragmentation techniques such as digestion with restriction endonucleases or structure-specific endonucleases, and specific chemical cleavage.

### Validation of a Nucleic Acid Sequence without Sequencing

The methods of the present invention are useful to validate the sequence of a nucleic acid such as a cDNA cloned into a plasmid or other vector, without de novo sequencing, e.g., Sanger or hybridization sequencing. FT-ICR MS, as disclosed in the application, is focused at analyzing cDNAs for mass variations compared to appropriate reference sequence cDNAs. With a draft in hand of the primary DNA sequence of the human genome, one of the next large undertakings in biology is the assembly of a complete set of full-length cDNAs and their variants for all genes. This is an essential step in understanding the function of all genes as well as a starting point for the development of the next generation of biotherapeutics and target-specific small molecule drugs. While the existing sequence information derived from the human genome project and the EST sequencing projects enables accurate predictions to be made of the primary sequence of most full-length cDNAs, the assembled cDNAs still must be sequence validated to determine subtle genetic alterations, e.g. point mutations, genetic polymorphisms, splicing variants, etc., that may not be readily discerned by common, high-throughput, inexpensive laboratory methods such as gel electrophoresis. While Sanger sequencing of partial or full-length cDNAs will detect any variations at the molecular level, this strategy is prohibitively expensive and an unnecessary tact given that most of the sequence for each cDNA in question will be invariant from that predicted based on the relevant reference cDNA sequence.

Nucleic acids to be sequence validated can be from any source, including genomic DNA, cDNA, synthetic DNA, and RNA. The nucleic acids can also be amplified by PCR; templates for PCR include previously isolated cDNA clones, cloned libraries of cDNAs, and RNA derived from appropriate cell or tissue sources which is reverse transcribed into cDNA. In general, all PCR primers will be preferably positioned in unique, non-repetitive sequence stretches and anneal to their respective complementary strand at similar thermodynamic stability to enable amplification conditions to be uniform for all amplicons. For amplifying cDNAs from clones, primers can be located either in the vector or within the cDNA insert itself. Generating cDNA amplicons from RNAs isolated from cells or tissues (e.g., from pathological specimens and adjacent unaffected tissue) will necessitate that the primers be located within the cognate cDNA that results from the RT reaction. In some embodiments wherein the nucleic acid of interest cannot be efficiently amplified in a single reaction, a series of minimally overlapping amplicons (e.g., each 2 kb in length) encoding relevant aspects of the cDNA, e.g. 5′ UTR and ORF, will be generated individually or simultaneously as part of one or more multiplex PCR reactions. Amplicons will be generated by PCR using a high fidelity, thermostable DNA polymerase or fragments thereof (Klenow-like), e.g. PfuI DNA polymerase, which lack both non-templated nucleotide polymerization activity and 3′ exonuclease activity. In some embodiments, the size of the nucleic acids to be validated may be greater than 10 kilobases.

Nucleic acids, including putative full-length or partial cDNA-derived amplicons, whose size is within the resolving range of FT-ICR will be analyzed for mass variation without fragmentation. The present invention anticipates mass analysis of unfragmented nucleic acids of 200 bases or more, and contemplates analyzing larger nucleic acids (e.g., nucleic acids greater than 250, 300, 400, 500, 750 and 1000 bases in length). Nucleic acids can be analyzed either individually or as mixtures with other nucleic acids that are also within the resolving range of FT-ICR. Preparation of mixtures of nucleic acids is particularly useful when PCR, including multiplexed PCR, is used to generated nucleic acids for validation. Those nucleic acids whose size is beyond the resolving range of FT-ICR will be fragmented prior to analysis for mass variation. Fragmentation of nucleic acids will be done using one or more sequence specific DNA hydrolases, e.g. restriction enzymes, universal enzymes, etc., whose recognition site is small and therefore occurs frequently in double stranded DNA. Examples include simple four base cutters like AluI, discontinuous four base cutters like HinFI, GANTC, and other restriction enzymes with slightly larger restriction sites due to sequence degeneracy, e.g. PspGI, which cuts at the sequence CCWGG. Based on the predicted frequency of occurrence of restriction enzyme sites within a designated nucleic acid, the nucleic acids will be digested using one or more restriction enzymes to cleave the DNA such that the sizes of the expected restriction enzyme fragments are within the range of resolution and can be unambiguously distinguished from other fragments within the digest by fragment mass determinations utilizing a mass spectrotrometer (MS), preferably utilizing ESI-FTICR, that determine M/Z with high range, resolution, and accuracy e.g. ≦200 bp, 30,000 (M/ΔM) and >0.01%, respectively.

To validate the sequence of a test nucleic acid relative to its corresponding reference nucleic acid sequence, the nucleic acids, PCR amplicons or restriction enzyme fragments derived from the nucleic acids are analyzed by MS to determine first, the M/Z value for each resolvable amplicon/RE fragment and then, the mass for each nucleic acid or restriction enzyme fragment as appropriate. The mass determination for each nucleic acid or restriction enzyme fragment is compared to the expected values from the corresponding nucleic acid reference sequence. The nucleic acid reference sequence may be present in a database containing known or predicted nucleic acid sequences. In those instances when mass analysis by ESI-FTICR of one or more test nucleic acids or restriction enzyme fragments derived from a test nucleic acid is identical to that expected for a nucleic acid or a restriction enzyme fragment derived from the reference sequence, the sequence of the test nucleic acid is validated. Alternatively, analyses that reveal mass differences between one or more test nucleic acids or restriction enzyme fragments and the corresponding reference nucleic acid denote variant nucleic acids having a sequence different than from the reference sequence. When a mass variant nucleic acid or a restriction enzyme fragment is identified, the variant nucleic acid or a restriction enzyme fragment is sequenced either completely or within an interval that will encompass the restriction enzyme fragment(s) of variant mass so as to determine the cause of the mass aberration at the molecular level. In some embodiments of the invention, once one or more regions containing one or more variant nucleic acid sequences are identified, those region(s) are selected for further mass spectral analysis, either by generating restriction enzyme fragments encompassing the regions or by amplifying sub-regions using PCR, or by other means described herein.

### Target Nucleic Acids

The target nucleic acid to which the methods of the invention are applied can be any gene or fragment thereof, a nucleic acid generated by PCR, a cDNA contained within a vector, or all or a portion of a chromosome. The target nucleic acid can be of any length that is capable of being acted upon by a separation means such as one or more restriction enzymes. Target nucleic acids can be, e.g., from about 200 bases to greater than 100,000 bases. No prior amplification or selection of the target nucleic acid is required to practice the methods of the present invention. Alternatively, the target nucleic acid is synthetic. The source of the nucleic acid is any nucleic acid-containing entity, including a whole organism, an organ, a tissue, a cell, a sub-cellular fraction, nucleic acids purified or obtained from biological materials and the like. The nucleic acid source can also be a non-biological material to which a biological material has been contacted, such as an article of clothing contacted with a body fluid, e.g., blood, saliva, tears, urine, perspiration, semen, or vaginal secretions.

### Fragmentation of Target Nucleic Acids

Fragmentation of a target nucleic acid results from contacting the target nucleic acid with one or more separation means, such that two or more double stranded nucleic acid fragments are generated from the test nucleic acid. In a preferred embodiment, the nonrandom length fragments generated by the methods of the present invention are of a size capable of being accurately measured by mass spectrometry. By way of non-limiting example, the fragment size is under 1,000 bases. The fragment size can also be under about 500, 200, 100, 75, 50, 20 or 10 bases. For purposes of this invention, fragmentation methods that produce a set of random length fragments are not desirable due to the limited reproducibility of such fragments, the limited information available from mass spectrometry analysis of such fragments, and the likelihood of spectral overlap from randomly generated fragments.

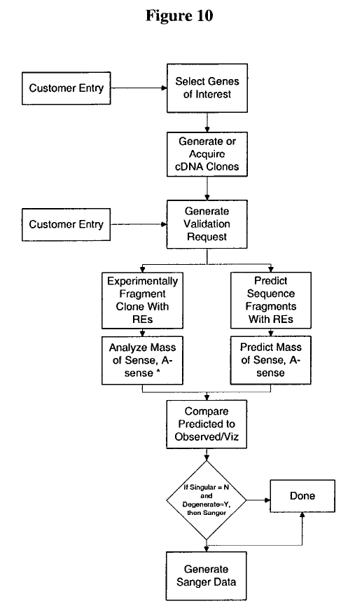

For analysis with mass spectrometry, a set of nonrandom length fragments is preferably generated ranging in length from 10-1000 bases, preferably from about 20 to about 200 bases in length. The range of lengths serves to better separate and resolve the fragment peaks in the resulting mass spectrum. Optional, subsequent iterations of the validation or polymorphism detection methods use progressively smaller length fragments. For example, a first set of nonrandom length fragments is generated ranging in length from 100 to 200 bases in length and analyzed using ESI-FITCR MS. A second set of nonrandom length fragments is then generated ranging in length from about 60 to about 100 bases in length and analyzed using ESI-FITCR MS. A third set of nonrandom length fragments is then generated ranging in length from about 20 to about 40 bases in length and analyzed using ESI-FITCR MS. A fourth set of nonrandom length fragments is then generated ranging in length from about 10 to about 20 bases in length and analyzed using ESI-FITCR MS. The resulting polymorphism-containing fragment is then sequenced by standard methods well known in the art. A schematic of a representative process is illustrated in FIG. 10. In this manner, a target nucleic acid 2,000 bases in length could be analyzed with a coverage of 3×, to a window of 20 base pairs on average by 4 iterations of the methods of the invention.

Fragmentation of target nucleic acids can be accomplished using a number of means, including cleavage with one or more DNA restriction endonucleases targeting specific sequences within double-stranded DNA, chemical cleavage at structure-specific and/or base-specific locations, polymerase incorporation of modified nucleotides that create cleavage sites when incorporated, and targeted structure-specific and/or sequence-specific nuclease treatment.

In embodiments of the present invention, the restriction enzymes used are Type II enzymes, which cut DNA at defined positions close to or within their recognition sequences and generally produce discrete restriction fragments and distinct gel banding patterns. The most common type II enzymes cleave DNA within their recognition sequences, e.g., Hha I, Hind III and Not I. Most Type II enzymes recognize DNA sequences that are symmetric because they bind to DNA as homodimers, but a few, (e.g., BbvC I: CCTCAGC) recognize asymmetric DNA sequences because they bind as heterodimers. Some enzymes recognize continuous sequences (e.g., EcoR I: GAATTC) in which the two half-sites of the recognition sequence are adjacent, while others recognize discontinuous sequences (e.g., Bgl I: GCCNNNNNGGC; SEQ ID NO: 3) in which the half-sites are separated.

Other type II enzymes useful in the present invention cleave outside of their recognition sequence to one side. These enzymes are usually referred to as “type IIs” and include, e.g., Fok I and Alw I. These enzymes are intermediate in size, 400-650 amino acids in length, and they recognize sequences that are continuous and asymmetric. They comprise two distinct domains, one for DNA binding, the other for DNA cleavage. They are thought to bind to DNA as monomers for the most part, but to cleave DNA cooperatively, through dimerization of the cleavage domains of adjacent enzyme molecules. For this reason, some type IIs enzymes are much more active on DNA molecules that contain multiple recognition sites. The use of type IIs enzymes is preferred in situations wherein non-type IIs enzymes cannot generate a suitable set of nonrandom length fragments, such as in cases of low-complexity DNA, genomic DNA with Alu or other repeats, or polynucleotide repeats (e.g., AAAAAAAAA).

Still other type II enzymes useful in the present invention, also called “type IV” enzymes, are large, combination restriction-and-modification enzymes, 850-1250 amino acids in length, in which the two enzymatic activities reside in the same protein chain. These enzymes cleave outside of their recognition sequences; those that recognize continuous sequences (e.g., Eco57 I: CTGAAG) cleave on just one side; those that recognize discontinuous sequences (e.g., Bcg I: CGANNNNNNTGC; SEQ ID NO: 4) cleave on both sides releasing a small fragment containing the recognition sequence. The amino acid sequences of these enzymes are varied but their organization are consistent. They comprise an N-terminal DNA-cleavage domain joined to a DNA-modification domain and one or two DNA sequence-specificity domains forming the C-terminus, or present as a separate subunit. When these enzymes bind to their substrates, they switch into either restriction mode to cleave the DNA, or modification mode to methylate it.

In embodiments of the present invention, multiple rounds of nucleic acid fragmentation and mass spectral analysis are performed, in which the size of the fragmented nucleic acids decrease with each successive round of fragmentation. Multiple restriction enzymes are useful to generate nucleic acid fragments of specific, pre-determined lengths that maximize resolution of the mass spectrometry.

The double stranded nucleic acid fragments derived from the fragmentation process can be used directly in mass spectrometry without purification. In some embodiments, the fragmented nucleic acids can be purified. In preferred embodiments, the molecular masses of essentially all of the nucleic acid fragments generated by fragmentation are determined. As such it is generally unnecessary to remove any nucleic acid fragments prior to mass determination.

### Mass Spectrometry of Fragmented Double Stranded Nucleic Acids

Methods of conducting mass spectrometric analysis of high molecular weight molecules such as nucleic acid molecules and polypeptides are known in the art. See, e.g., Liu, C. et al., Anal. Chem. 1998, Vol. 70(9): 1797-1801; Yang, L. et al., Anal. Chem. 1997, Vol. 70(15): 3235-3241; Muddiman, D. C. et al. Anal. Chem. 1997, Vol. 69(8): 1543-1549; Muddiman, D. C. et al. Anal. Chem. 1996, Vol. 68(21): 3705-3712; Aaserud, D. J. et al., J. Am. Soc. Mass Spectrom. 1996 Vol. 7: 1266-1269; Winger, B. E. et al., J. Am. Soc. Mass Spectrom. 1993 Vol. 4: 566-577. The preferred types of mass spectrometry used in the invention include ion cyclotron resonance mass spectrometry, electrospray ionization fourier transform ion cyclotron resonance (ESI-FTICR) mass spectrometry, matrix-assisted laser desorption ionization (MALDI) mass spectrometry, quadropole ion trap mass spectrometry, magnetic/electric sector mass spectrometry and time-of-flight mass spectrometry. A preferred method of mass spectrometry is ESI-FTICR.

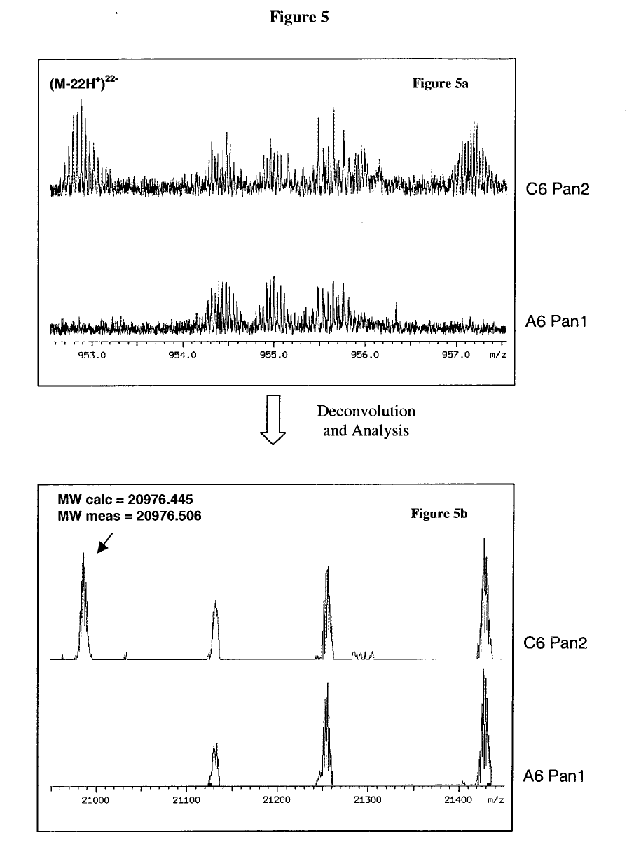

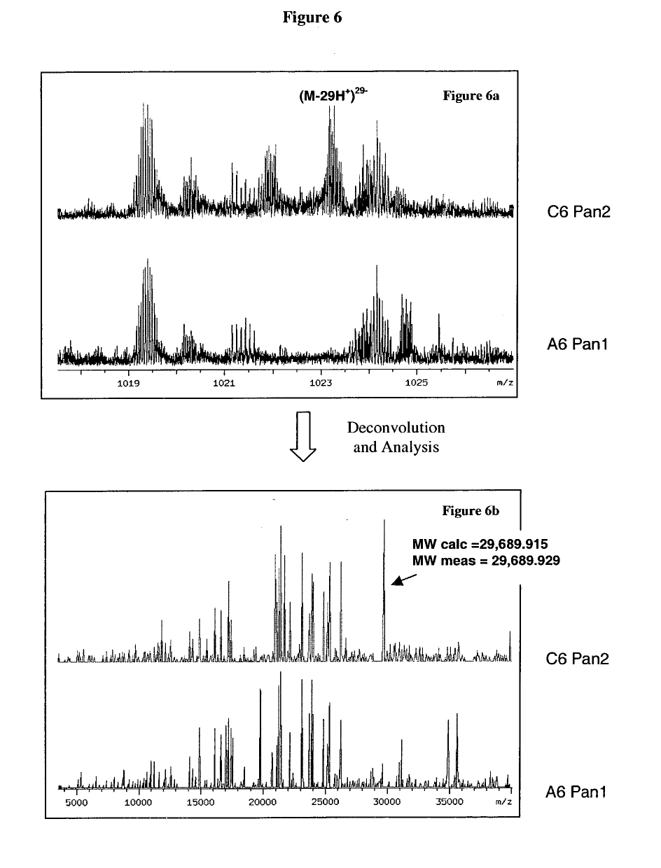

Existing mass spectrometric instrumentation in the case of ESI-FITCR MS optimally has a mass accuracy of <0.5 Da, 20 times what is necessary for detecting a single base change in a 50-base long single-stranded DNA fragment. Continued advances in mass spectrometric instrumentation will also push this range higher. Examples of the resolving capabilities of ESI-FITCR MS are displayed in FIGS. 5 and 6.

In one aspect of this invention the methods are conducted to accurately determine the masses of a set of nonrandom length fragments and this data is correlated to a reference set of fragments to determine the presence or absence of a polymorphism, followed by optional characterization of any polymorphism present. An advance of the present invention is the ability to perform mass spectrometric determination of the members of a set of double-stranded nonrandom length fragments, optionally in an iterative manner, such that the sequence validity of a nucleic acid can be determined without sequencing the entire nucleic acid.

The preferred method of mass spectrometry is ESI-FITCR MS, in part because of the ability to determine the molecular masses of both strands of double stranded DNA simultaneously. ESI is the more gentle ionization procedure, producing a denatured but intact positive and negative strands. Other MS techniques like MALDI are less preferred owing to the complex fragmentation patterns and the lack of resolving power of all the mass fragments.

### Internal Mass Calibrants

Mass spectrometers are typically calibrated using analytes of known mass. A mass spectrometer can then analyze an analyte of unknown mass with an associated mass accuracy and precision. However, the calibration, and associated mass accuracy and precision, for a given mass spectrometry system (including MALDI-TOF MS) can be significantly improved if analytes of known mass are contained within the sample containing the analyte(s) of unknown mass(es). The inclusion of these known mass analytes within the sample is referred to as use of internal calibrants. External calibrants, i.e. analytes of known mass that are not mixed in with the set of nonrandom length fragments of unknown mass and simultaneously analyzed in a mass spectrometer, are analyzed separately. External calibrants can also be used to improve mass accuracy, but because they are not analyzed simultaneously with the set of fragments of unknown mass, they will not increase mass accuracy as much as internal calibrants do. Another disadvantage of using external calibrants is that it requires an extra sample to be analyzed by the mass spectrometer. For MALDI-TOF MS, generally only two calibrant molecules are needed for complete calibration, although sometimes three or more calibrants are used. For ESI-FTICR, the abundance of internal calibrants is sufficient, although a high molecular weight calibrant is often added to help with the automatic detection of peaks in the samples. All of the embodiments of the invention described herein can be performed with the use of internal calibrants to provide improved mass accuracy.

Using the methods described herein, one can obtain a mass spectrum with numerous mass peaks corresponding to the set of nonrandom length fragments of the gene or target nucleic acid under study. If no mutation is present in the target nucleic acid, all of the mass peaks corresponding to the nonrandom length fragments will be at mass-to-charge ratios associated with the set of NLFs from the wild type target nucleic acid. However, if the target nucleic acid contains a mutation, usually no more than one or two of the mass peaks will be shifted in mass, leaving the majority of mass peaks at unaltered locations. In a preferred embodiment of the invention, a self-calibration algorithm uses these unmutated or nonpolymorphic NLFs for internal calibration to optimize the mass accuracy for analysis of the NLFs containing a mutation, thus requiring no added calibrant(s), simplifying the calibration, and avoiding potential spectral overlaps. In a given sample, however, it will not be known a priori which mass peaks, if any, are altered or shifted from their expected masses for the wild type NLFs.

The self-calibration algorithm begins by dividing up the observed mass peaks into subsets, each subset consisting of all but one or two of the observed mass peaks. Each data subset has a different one or two mass peaks deleted from consideration. For each subset, the algorithm divides the subset further into a first group of two or three masses which are then used to generate a new set of calibration constants, and a second group which will serve as an internal consistency check on those new constants. The internal consistency check begins by calculating the mass difference between the m/z values calculated for the second group of mass peaks and the values corresponding to reasonable choices for the associated wild-type NLFs. The internal consistency check can thus take the form of a chi-square minimization where the key parameter is this mass difference. The algorithm finds which data subset has the lowest sum of the squares of these mass differences resulting in a choice of optimized calibration constants associated with group one of this data subset.

After new self-optimized calibration constants are obtained, the mass-to-charge ratios are determined for the mass peaks omitted from the data subset; these are the nonrandom length fragments suspected to contain a mutation. The differences from the observed mass peaks for the wild type NLFs are then used to determine whether a mutation has occurred, and if so, what the nature of this mutation is (e.g. the exact type of deletion, insertion, or point mutation). This self-calibration procedure should yield a mass accuracy of approximately 1 part in 10,000.

### Database Generation and Validation System

The present invention also provides a system for validating a target double stranded nucleic acid molecule and optionally identifying unique features (i.e., mutations) therein. The validation system is based on a database of fragments of predicted, wild type nucleic acid molecules against which the fragments of the target double stranded nucleic acid molecule is compared. The flow diagram in FIG. 10 describes an embodiment of the validation system applied to one embodiment of the invention, validation of a cDNA sequence. The system initially comprises having a user make a selection of one or more genes of interest, followed by the acquisition of or creation of cDNA clone samples for the selected gene(s). Upon receiving and recording a request to perform a validation for the cDNA clone samples, the system branches into two activities. In the first activity, cDNA samples are fragmented using fragmentation means, e.g., by contact of cDNA with various restriction enzymes, and masses are determined for sense and anti-sense strands of DNA. In the second activity, in silico calculations are performed to predict cDNA fragmentation based upon the desired genes and the restriction enzyme(s) to be applied, resulting in algorithmic calculations of the masses for sense and anti-sense strands of DNA. After the first and second activities have been carried out, the resulting data sets are merged to compare the observed results with the predicted results. Gene matching and validation conclusions can then be drawn from the comparisons.

### Building Reference Database

This invention also provides a reference database of wild type nucleic acid sequences. The reference database can be generated from the available nucleic acid sequence databases such as Genbank, EMBL, DDBJ, PDB, GSS, BDGP (the drosophila genome project), the CuraGen GeneCalling® database and the Celera Discovery System. Alternatively the database can be generated from experimental sequence analysis of wild type genes. Preferably, the database of the invention is designed to be non-redundant in order to simplify the downstream analysis, which can be confused if multiple, redundant entries are found in the database.

The flow diagram in FIG. 11 depicts one such procedure for developing a reference database. The cDNA Reference Database (Ref DB) is a database of putative genes and predicted fragment information that would be expected by experimentally applying separation means, such as restriction enzymes (REs), to cDNA samples. The Ref DB is used during the clone validation to compare observed cDNA (digested) fragments against predicted fragments. The process for building the Ref DB begins with a selection of genes for which fragment predictions will be carried out. If information about gene is found (is available in public or commercial sequence databases), a search is performed to find cDNA sequence information for the gene. If cDNA sequence information is located, the cDNA sequence is captured and the gene will be marked to indicate that real cDNA information exists. If cDNA sequence information is not found, the genomic DNA (gDNA) sequence information is obtained, and cDNA will be predicted from the gDNA, using an algorithm to predict introns and exons, and then assembling the exons into a predicted cDNA sequence. Following the cDNA prediction process, the gene will be marked as predicted cDNA.

After the cDNA information has been determined for a gene, that information is stored in the Ref DB. Then, applying desired sets of REs, a process predicts the digested fragments that would result from experimentally applying the REs to a real cDNA sample (see “Predict RE-Cleaved Fragments” section for more details). Each predicted fragment is stored in the Ref DB with references to the source cDNA and the REs that were used in the prediction.

From the database, an optimal set (or global set) of separation means, preferably REs are selected to generate overlapping fragments from which the entire target sequence can be covered. For each cut fragment, knowing the overhangs on the 3′ and 5′ ends allows for the exact determination of the composition of each strand. The resulting single strand mass can be directly computed from the composition multiplied by the monoisotopic molecular weight of each nucleotide:

A=331

C=307

G=347

T=322

Commercial and public domain software, such as Nucleotide Mass Calculator, (University of Washington), is available for this purpose.

Once the database is generated, actual sets of test nucleic acid fragments can be generated by contacting the sample with the identical fragmentation means used to generate the database fragment set. The test nucleic acid fragment set is then subject to mass analysis, preferably by mass spectrometric methods, to determine the mass ranges of the test nucleic acid fragment set. Mass range data can be stored as numerical values in a table or displayed in a graphical representation. Comparison of data from the generated test set with the fragment database set allows for validation of the sequence of the test nucleic acid molecule. A variety of statistical approaches can be applied in order to select which table of predicted RE fragments masses is the best fit, including non-linear regression analysis, neural network-type clustering, or a Bayesian analysis.

### Predicting RE-Cleaved Fragments

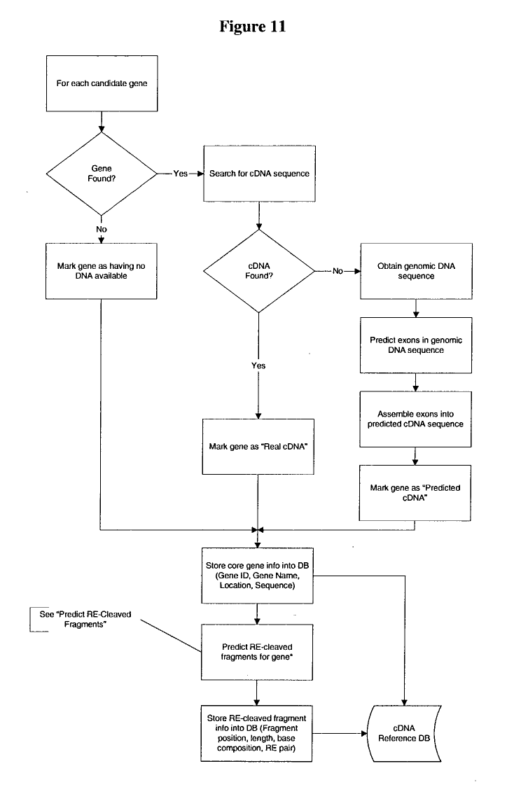

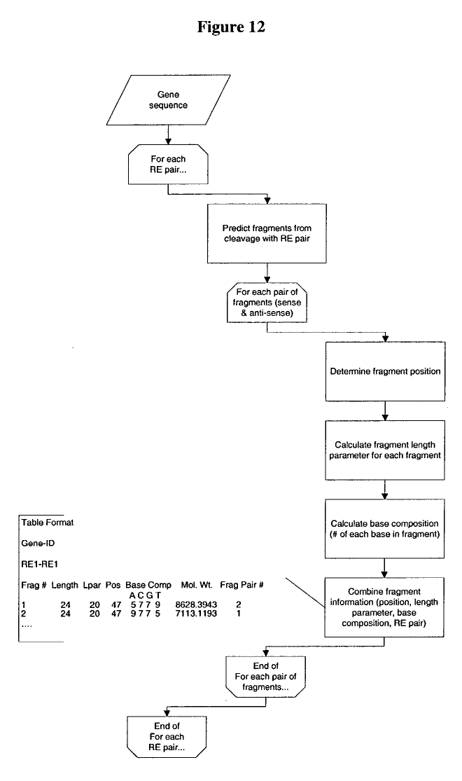

The invention also provides a method for predicting cleaved nucleic acid fragments, which process predicts the results of experimentally combining sets of REs with a particular nucleic acid sample, in particular a cDNA sample. In the embodiment of the method shown in FIG. 12, the prediction process begins with the gene sequence for the cDNA, and for each desired RE, predicts the cleavage sites and the resulting fragments that would be expected in experimental work, both for the sense and anti-sense strands of the DNA. For each fragment predicted, the user can determine the fragment starting position, length, nucleotide base composition, and molecular weight. All of the predicted fragment information is stored in the Ref DB.

### Generate Fragments Experimentally from Clones

The invention also provides a system for experimentally generating fragments from cDNA clone samples. As depicted in the embodiment shown in FIG. 13, a user logs into the system and reviews the queue for sample processing requests, and then receives incoming cDNA samples. In the system, the samples are advanced to the queue for performing RE separation laboratory work, and then the samples are stored in a refrigeration unit until the experimental work will begin. The RE fragmentation laboratory process consists of three steps. The first step is focused on preparing reagent plates, consisting of RE pairs and buffer. The second step consists of combining the contents of the reagent plates with a plate that contains the cDNA sample. The third step is to let the combined sample/reagent plate sit for several hours (generally overnight) at an appropriate temperature, e.g., 37° centigrade. The final step is conducted in a manner to allow the RE pairs to cleave the cDNA sample and result in fragmentation of the cDNA. Following the lab work, the samples are ready for mass spectrometry, which can be done by the user or sent to a supplier of mass spectrometry sequencing services.

### Generate Fragment Data

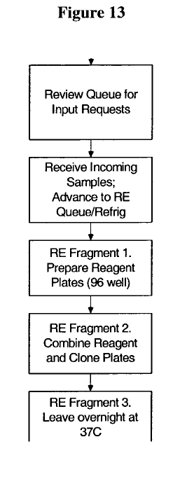

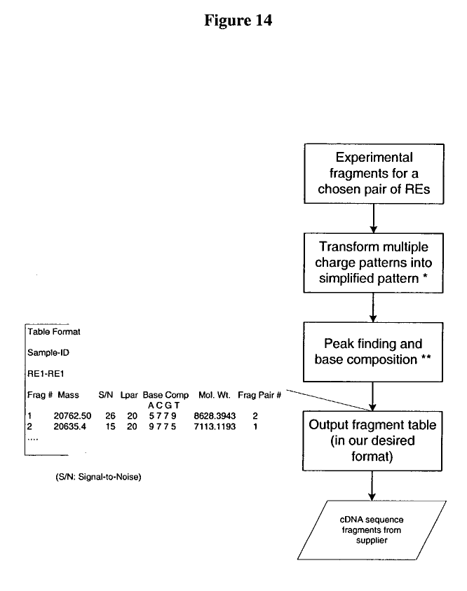

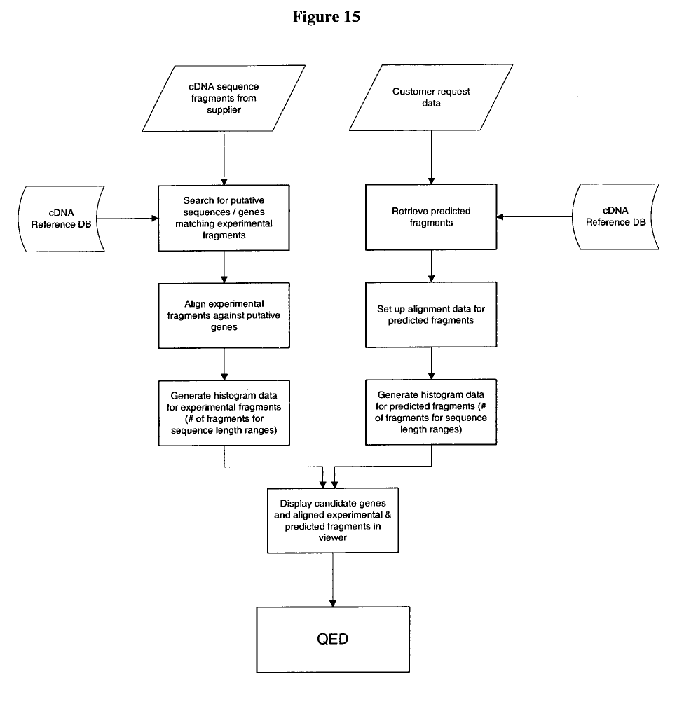

The purpose of the mass spectrometry sequencing aspect of the invention is to generate observed fragment data that can be used to identify the gene represented by the nucleic acid, in particular the cDNA, sample. Thus, an additional aspect of this invention is the provision of nucleic acid fragment data, in particular gene fragment data for genes of interest. As depicted in the embodiment shown in FIG. 14, after the mass spectrometry sequencing work has been performed, a set of experimental fragments will result for each chosen RE pair. The initial data consists of multiple charge patterns. The next step is to transform the data into a simplified pattern such that peak finding can be performed for each fragment and the base composition can be determined for the fragment based upon the number of bases and the molecular weight of the fragment. With determinant fragment data established, the fragment sets can be packaged by, e.g., cDNA sample and RE.

### Comparing Observed Experimental and Predicted Fragments

This invention further provides a system for comparing observed experimental fragment mass data with the mass data generated from the method for producing predicted fragments of the nucleic acid molecule of interest, preferably a gene. As depicted in the embodiment shown in FIG. 14, following experimental and in silico procedures to determine observed and predicted fragmentation for a given nucleic acid, preferably cDNA, sample and desired REs, several steps occur to allow the observed and predicted fragments to be compared. First, the observed are aligned against putative genes using one or more local sequence alignment tools such as BLAST and Smith-Waterman. Then, a histogram is generated for the observed fragments based upon the number of fragments that fall within a set of fragment length ranges. Concurrently, predicted fragments for the same cDNA are retrieved from the Ref DB, aligned, and a histogram is generated for the predicted fragments based upon the number of fragments that fall within a set of fragment length ranges. Finally, the observed and predicted fragments, along with their respective histograms are presented to a user in a viewer tool. The viewer tool allows the user to visually examine the match between observed fragments and predicted fragments. Using the viewer tool, in the vast majority of cases, the user will be able to determine whether the experimental data sufficiently matches the predicted data to infer the identity of (validate) the cDNA sample.

### Clone Validation System

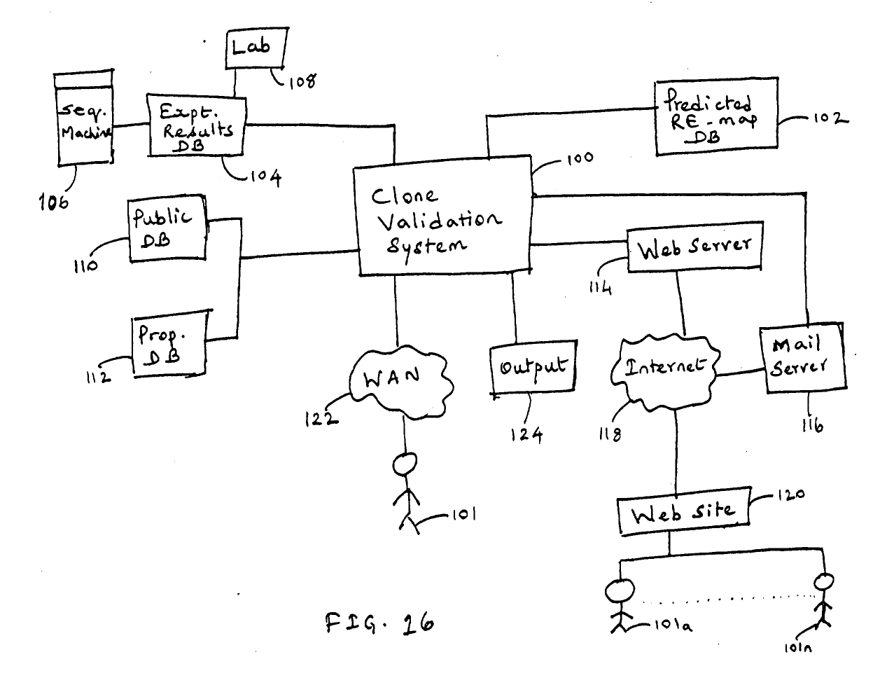

This invention further provides a clone validation system. As illustrated in FIG. 16, a clone validation system 100 may include or otherwise access data from, for example, predicted restriction map database 102 and experimental results database 104. Predicted restriction map database 102 may include predicted restriction maps of one or more nucleic acid sequence fragments (e.g., cDNA, portion of genomic DNA, etc.,). Experimental results database 104 may include, for example, experimentally observed data of restriction maps of one or more nucleic acid sequence fragments (e.g., cDNA, portion of genomic DNA, etc.,). The restriction maps of both predicted restriction map database 102 and experimental results database 104 may include a plurality of cleaving sites for one or more restriction endonucleases (e.g., EcoRI). In one embodiment, the cleaving sites may be organized for sensed strands of one or more DNA fragments. In another embodiment, the cleaving sites may be organized for anti-sensed strands of one or more DNA fragments. In yet another embodiment, the cleaving sites may be organized for the pair of strands of one or more DNA fragments. Both predicted restriction map database 102 and experimental results database 104 may also include, for example, but not limited to an identification number, base composition (e.g, proportion of guanine), and molecular weight for each of the stored nucleic acid sequence fragments corresponding to the restriction map.

In one embodiment, the experimental database 104 may be coupled to a sequencing machine 106. In another embodiment, the experimental database 108 map be coupled to a plurality of equipments in a laboratory 108.

According to another aspect of the invention, clone validation system 100 may be coupled to or otherwise access data from one or more public databases (e.g., GenBank) and/or one or more proprietary databases (e.g., Celera Genome Database).

Clone validation system 100 may also be coupled to web server 114 and mail server 116. Both web server 114 and mail server 116 may obtain data from clone validation system 100, process the data and enable one or more remote users 101a-n to access the processed data through a web site 120. In some embodiments, mail server may enable one or more remote users to access the processed data through a non-web based electronic mail system (not shown in figure). According to one embodiment, clone validation system may be coupled to wide area network (WAN) 122 and local area network (LAN) (not shown in figures). Clone validation system 100 may also be coupled to one or more output means 124 (e.g., display). A user 101 may obtain results using the one or more output means 124.

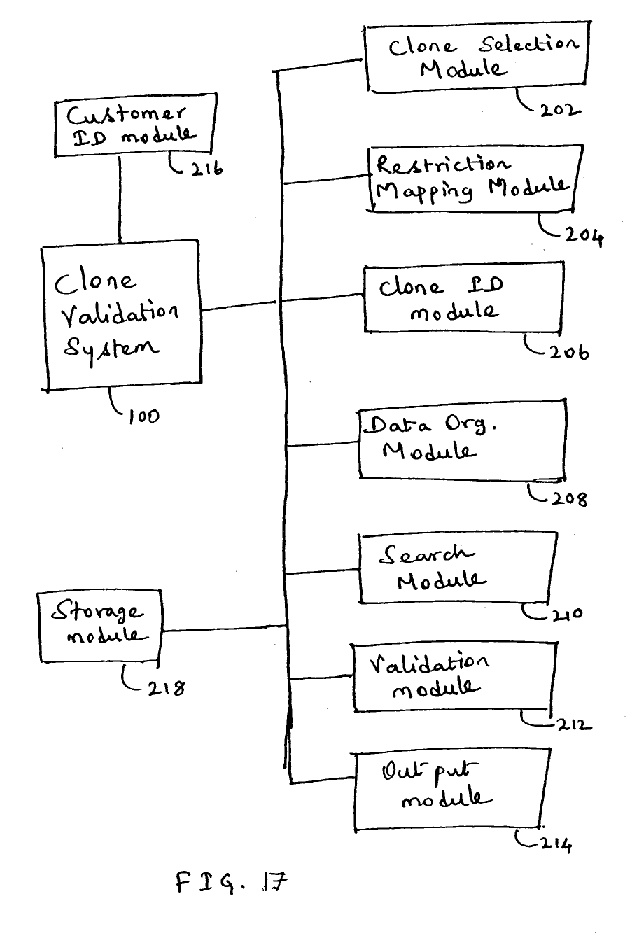

According to another aspect of the invention, as illustrated in FIG. 17, clone validation system 100 may include a plurality of modules including, for example, clone selection module 202, restriction mapping module 204, clone identification module 206, data organization module 208, search module 210, validation module 212, output module 214, customer identification module 216, and storage module 218.

Clone selection module 202 may enable a user to select one or more genes and identify nucleic acid sequence fragments corresponding to the user selected genes. Restriction mapping module 204 may predict one or more cleaving sites for one or more separation means in the nucleic acid sequence fragments corresponding to the user selected genes. In some embodiments, restriction mapping module 204 may predict one or more cleaving sites for one or more separation means specified by a user. This prediction may be performed by one or more user selectable algorithms (e.g., neural network algorithm, etc.,) in the system 100. In a preferred embodiment, mass determination module 205 (not shown in figure) is included to calculate the mass of the fragments corresponding to the user selected genes using one or more mass determining algorithms.

Clone identification module 206 may enable a user to assign an identification code (e.g., an alpha numeric code) for nucleic acid sequence fragments corresponding to the user selected genes. Clone identification module 206 may also identify position of restriction enzyme binding sites, and calculate composition of As, Ts, Gs, and Cs and molecular weight for nucleic acid sequence fragments corresponding to the user selected genes.

Data organization module 208 may organize the data, for example, identification code, molecular weight, etc., in a user specified manner. The organized data may be presented to a user through a display of output means 124.

Search module 210 may enable a user to search for unique nucleic acid sequences associated with the sequences of the user selected genes. In one embodiment, search module 210 may enable a user to search for nucleic acid sequences, preferably cDNA sequences, associated with the user selected genes. In another embodiment, search module 210 may enable a user to search for genomic sequence fragments including introns, and exons associated with the user selected genes. In yet another embodiment, search module 210 may enable a user to search for regulatory sequences associated with the user selected genes.

Validation module 212 may validate the nucleic acid sequences of the user selected genes by evaluating the predicted data for cleaving portions with experimentally observed data for cleaving portions. In one embodiment, this evaluation may be performed by, for example, probabilistic modeling of a predicted data versus experimental data. In another embodiment, this evaluation may be performed by one or more user selectable validation algorithms in the system 100. In one embodiment, a validation algorithm in the system 100 may correspond to a plurality of processes, for example, but not limited to obtaining a user requests for validation of one or more clones (e.g., genes, sequence fragments), predicting restriction sites in the one or more clones, retrieving experimental results of the restriction sites, and statistically analyzing predicted restriction sites with experimental results of the restriction sites. In some embodiments, the validation module 212 may validate the nucleic acid sequences corresponding to the user selected genes by evaluating the predicted mass of the nucleic acid fragments corresponding to the user selected genes against the experimentally observed mass data stored in the experimental results database 104. The system 100 may determine the divergence in the nucleic acid fragments corresponding to the user selected genes based this evaluation and identify the fragments that may need further validation by sequencing.

Output module 214 may output the results of the validation and enables a user to identify unique features, for example, but not limited to single nucleotide polymorphisms (SNPs), micro-satellites, mini-satellites, etc. In some embodiments, output module 214 may enable a user to identify candidate genes for the nucleic acid sequences corresponding to the user selected genes.

Storage module 218 may store the results of search, validation, and output for the nucleic acid sequences corresponding to the user selected genes. In some embodiments, a user may be able to store predicted restriction sites for each of the nucleic acid sequence fragments analyzed by the system 100.

Customer identification module 216 may store user data, including, for example, user log-in, password etc., of a plurality of users using clone validation system 100. Customer identification module may also track activities of a user, for example, time logged-in, time logged-out, duration of usage of clone validation system, etc.

Finally, the invention provides a method for medical decision making based on the presence or absence of a gene of interest in the test double stranded nucleic acid molecule. Such medical decision making can comprise diagnosis of a genetic-based disorder and chromosomal aneuploidy or genetic predisposition to disease state.

The following examples are intended only to illustrate the present invention and should in no way be construed as limiting the subject invention.

### EXAMPLE 1

### cDNA Validation

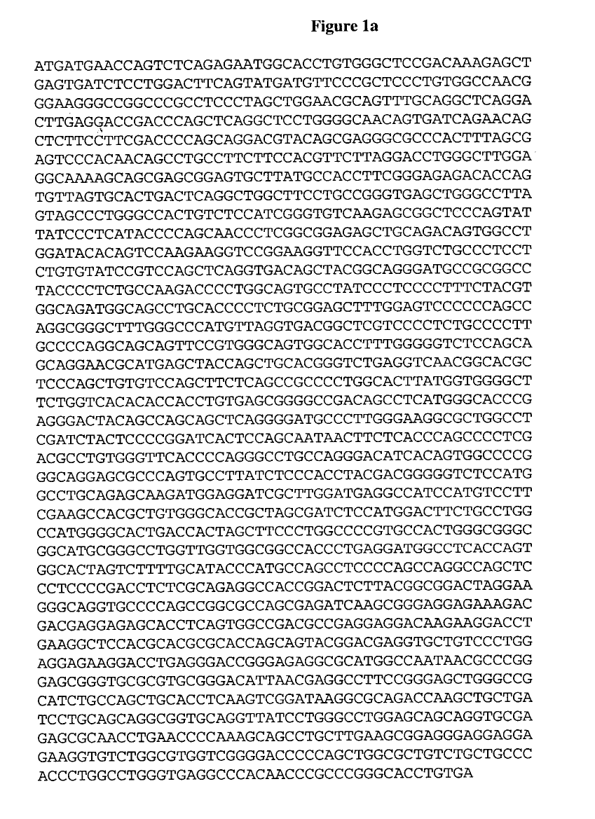

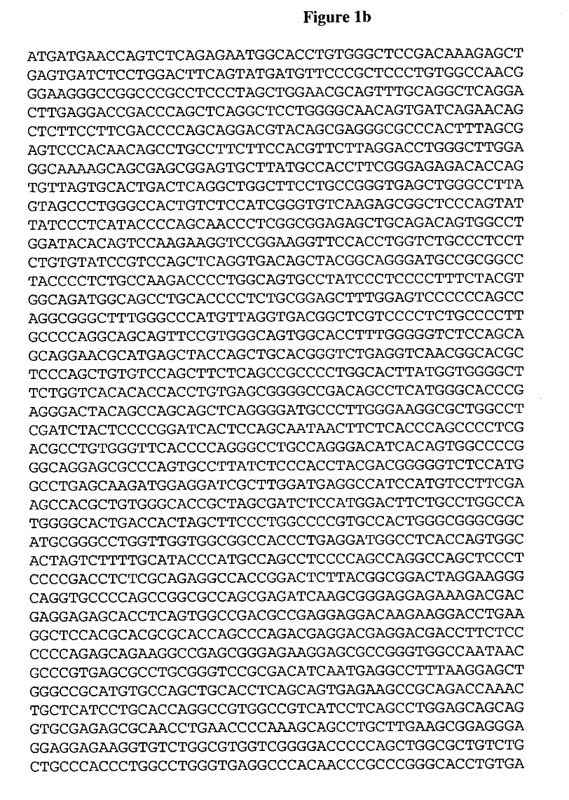

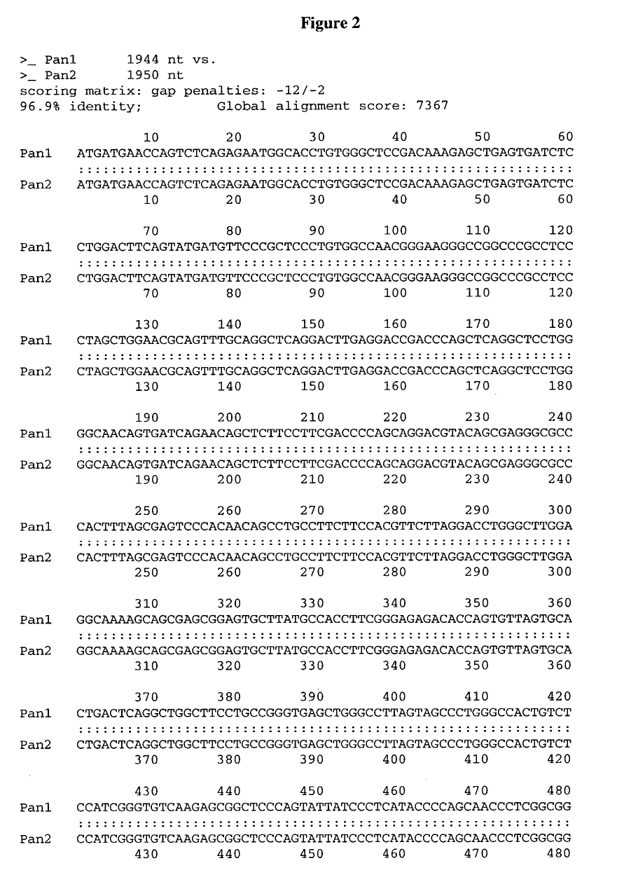

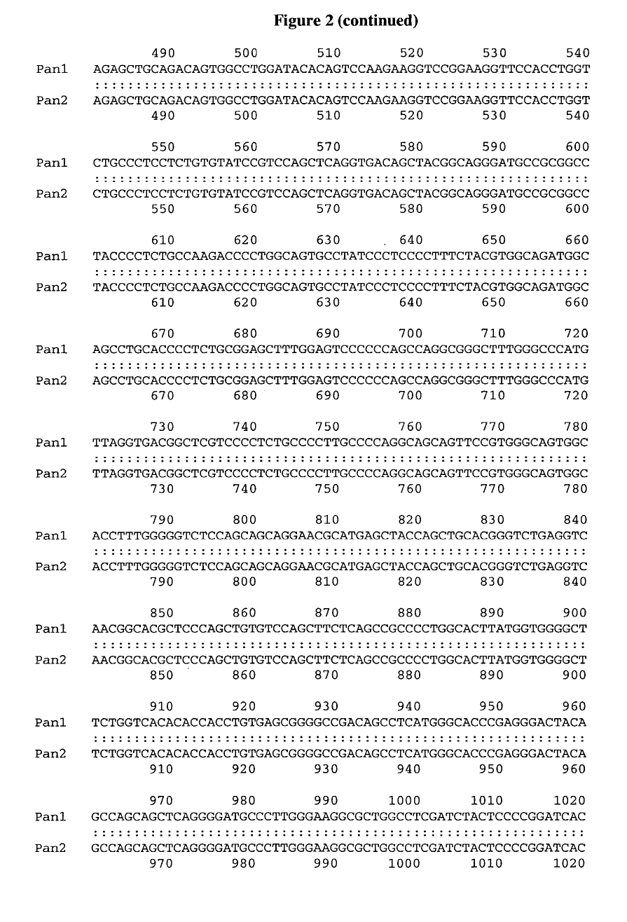

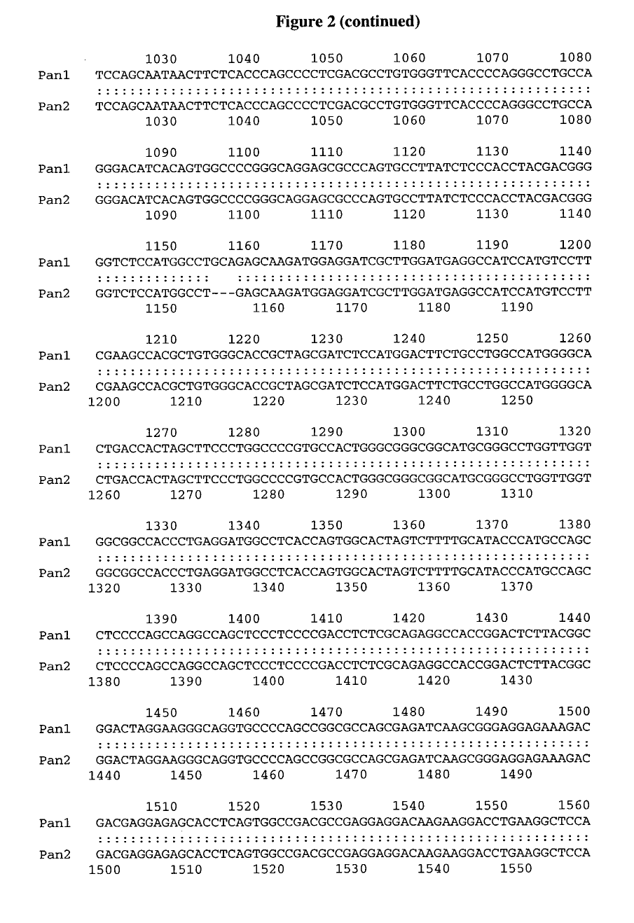

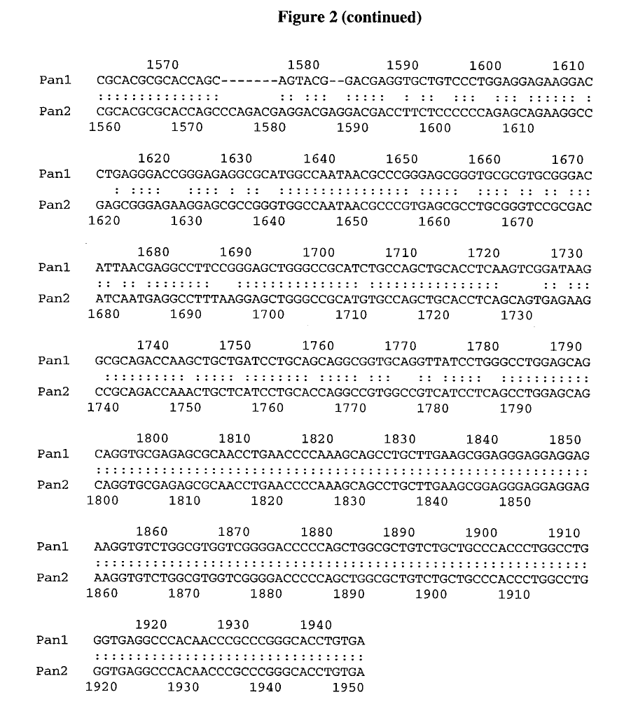

This example describes ESI-FITCR analysis of restriction digested Pan1 and Pan2 Nucleic Acids. cDNAs encoding the Pan1 transcription factor and a known, Pan1-like cDNA sequence variant Pan2 are provided in FIG. 1 along with a pairwise alignment of the two sequences in FIG. 2. (See, German, M. et al., Molecular Endocrinology 1991, Vol. 5: 292-299). As shown in FIG. 2, Pan1 and Pan2 exhibit almost 97% sequence identity with complete identity from segments 1-1154, 1158-1575 and 1781-1944 bp using the Pan1 basepair coordinates. Consequently, the sequence divergence between Pan1 and Pan2 is focused in a 3 bp segment specified by bases 1155-1157 and a 205 bp segment specified by bases 1576-1780 of the Pan1 sequence. The regions of identity and divergence are identified using the methods of the present invention.

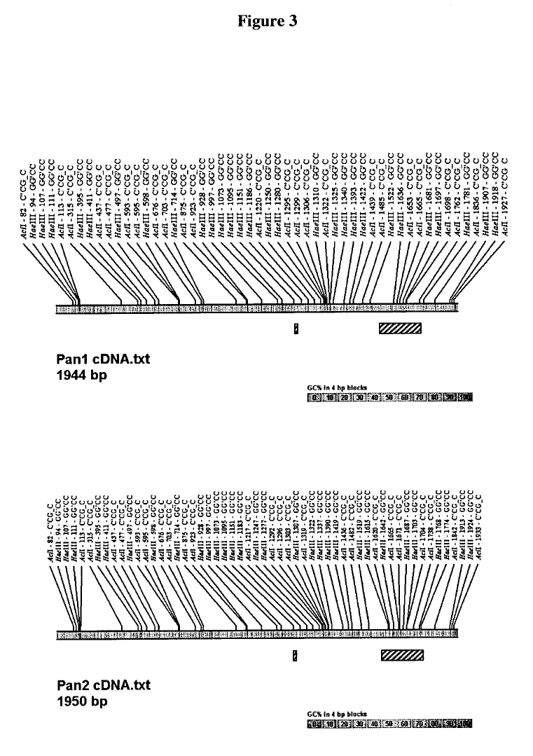

The Pan1 and Pan2 cDNAs are subjected to restriction enzyme digestion using AciI and HaeIII. A restriction enzyme map of each cDNA digested with AciI, and HaeIII is provided in FIG. 3. The region within each cDNA amplicon that encodes divergent sequence relative to its counterpart is shown with a cross hatched black rectangle below the depiction of the gene. Only those Pan2-derived restriction enzyme fragments that either span or partially overlap the specified divergent segment(s) will fail to validate the mass fragment pattern expected for a Pan1 sequence, and consequently, will result in one or more fragments with mass variation when compared to the Pan1 reference sequence. The same result will occur when comparing Pan1 -derived restriction enzyme fragments with fragments expected from a Pan2 reference sequence. Tables 1 and 2 provide a list of RE fragments resulting from single and double digestion of Pan1 and Pan2 cDNA with AciI (C′CGC) and HaeIII (GG′CC) and the expected molecular weights of the plus and minus strands for each fragment.

TABLE 1
Pan1 cDNA AciI + HaeIII Double Digestion Lookup Table

| | | Pan1 | Length | MW (monoisotopic) | |
|---|---|---|---|---|---|
| # | Ends | Coordinates | (bp) | Plus | Minus |
| 1 | (LeftEnd)-AciI | 1-82 | 82 | 25404.149 | 25893.217 |
| 2 | AciI-HaeIII | 83-94 | 12 | 3691.585 | 3140.528 |
| 3 | HaeIII-HaeIII | 95-107 | 13 | 4111.690 | 3955.625 |
| 4 | HaeIII-HaeIII | 108-111 | 4 | 1254.206 | 1254.206 |
| 5 | HaeIII-AciI | 112-113 | 2 | 596.102 | 1294.212 |
| 6 | AciI-AciI | 114-315 | 202 | 62242.135 | 62570.104 |
| 7 | AciI-HaeIII | 316-395 | 80 | 24844.005 | 23990.921 |
| 8 | HaeIII-HaeIII | 396-411 | 16 | 4950.798 | 4968.821 |
| 9 | HaeIII-AciI | 412-437 | 26 | 8023.304 | 8690.420 |
| 10 | AciI-AciI | 438-477 | 40 | 12131.975 | 12612.049 |
| 11 | AciI-HaeIII | 478-497 | 20 | 6309.041 | 5463.877 |
| 12 | HaeIII-AciI | 498-593 | 96 | 29602.802 | 30349.930 |
| 13 | AciI-AciI | 594-595 | 2 | 636.108 | 636.108 |
| 14 | AciI-HaeIII | 596-598 | 3 | 965.160 | 307.056 |
| 15 | HaeIII-AciI | 599-676 | 78 | 23682.810 | 25155.101 |
| 16 | AciI-AciI | 677-703 | 27 | 8338.351 | 8378.358 |
| 17 | AciI-HaeIII | 704-714 | 11 | 3482.552 | 2731.464 |
| 18 | HaeIII-AciI | 715-875 | 161 | 49554.986 | 50556.215 |
| 19 | AciI-AciI | 876-923 | 48 | 14785.380 | 14901.439 |
| 20 | AciI-HaeIII | 924-928 | 5 | 1623.264 | 885.147 |
| 21 | HaeIII-HaeIII | 929-997 | 69 | 21418.494 | 21244.406 |
| 22 | HaeIII-HaeIII | 998-1073 | 76 | 23106.746 | 23875.875 |
| 23 | HaeIII-HaeIII | 1074-1095 | 22 | 6822.121 | 6804.097 |
| 24 | HaeIII-HaeIII | 1096-1151 | 56 | 17211.779 | 17420.821 |
| 25 | HaeIII-HaeIII | 1152-1186 | 35 | 11000.806 | 10653.722 |
| 26 | HaeIII-AciI | 1187-1220 | 34 | 10414.689 | 11241.830 |
| 27 | AciI-HaeIII | 1221-1250 | 30 | 9225.482 | 8723.443 |
| 28 | HaeIII-HaeIII | 1251-1280 | 30 | 9219.494 | 9348.524 |
| 29 | HaeIII-AciI | 1281-1295 | 15 | 4607.741 | 5025.817 |
| 30 | AciI-AciI | 1296-1299 | 4 | 1294.212 | 1214.200 |
| 31 | AciI-AciI | 1300-1306 | 7 | 2200.361 | 2160.355 |
| 32 | AciI-HaeIII | 1307-1310 | 4 | 1294.212 | 596.102 |
| 33 | HaeIII-AciI | 1311-1322 | 12 | 3786.598 | 4280.717 |
| 34 | AciI-HaeIII | 1323-1325 | 3 | 965.160 | 307.056 |
| 35 | HaeIII-HaeIII | 1326-1340 | 15 | 4655.764 | 4646.752 |
| 36 | HaeIII-HaeIII | 1341-1393 | 53 | 16142.619 | 16631.705 |
| 37 | HaeIII-HaeIII | 1394-1422 | 29 | 8796.425 | 9156.481 |
| 38 | HaeIII-AciI | 1423-1439 | 17 | 5208.849 | 5946.966 |
| 39 | AciI-AciI | 1440-1485 | 46 | 14343.361 | 14111.243 |
| 40 | AciI-HaeIII | 1486-1522 | 37 | 11670.946 | 10602.676 |
| 41 | HaeIII-HaeIII | 1523-1636 | 114 | 35600.857 | 34860.539 |
| 42 | HaeIII-AciI | 1637-1653 | 17 | 5266.879 | 5888.937 |
| 43 | AciI-AciI | 1654-1665 | 12 | 3796.604 | 3654.603 |
| 44 | AciI-HaeIII | 1666-1681 | 16 | 5032.839 | 4267.687 |
| 45 | HaeIII-HaeIII | 1682-1697 | 16 | 4991.799 | 4929.810 |
| 46 | HaeIII-AciI | 1698-1698 | 1 | 307.056 | 965.160 |
| 47 | AciI-AciI | 1699-1762 | 64 | 19747.232 | 19822.192 |
| 48 | AciI-HaeIII | 1763-1781 | 19 | 5952.954 | 5201.866 |
| 49 | HaeIII-AciI | 1782-1836 | 55 | 17045.813 | 17582.806 |
| 50 | AciI-HaeIII | 1837-1907 | 71 | 22161.566 | 21121.423 |
| 51 | HaeIII-HaeIII | 1908-1918 | 11 | 3491.563 | 3340.550 |
| 52 | HaeIII-AciI | 1919-1927 | 9 | 2691.457 | 3522.558 |
| 53 | AciI-(RightEnd) | 1928-1944 | 17 | 5249.851 | 4671.759 |

TABLE 2
Pan2 cDNA AciI + HaeIII Double Digestion Lookup Table

| | | Pan2 | Length | MW (monoisotopic) | |
|---|---|---|---|---|---|
| # | Ends | Coordinates | (bp) | Plus | Minus |
| 1 | (LeftEnd)-AciI | 1-82 | 82 | 25404.149 | 25893.217 |
| 2 | AciI-HaeIII | 83-94 | 12 | 3691.585 | 3140.528 |
| 3 | HaeIII-HaeIII | 95-107 | 13 | 4111.690 | 3955.625 |
| 4 | HaeIII-HaeIII | 108-111 | 4 | 1254.206 | 1254.206 |
| 5 | HaeIII-AciI | 112-113 | 2 | 596.102 | 1294.212 |
| 6 | AciI-AciI | 114-315 | 202 | 62242.135 | 62570.104 |
| 7 | AciI-HaeIII | 316-395 | 80 | 24844.005 | 23990.921 |
| 8 | HaeIII-HaeIII | 396-411 | 16 | 4950.798 | 4968.821 |
| 9 | HaeIII-AciI | 412-437 | 26 | 8023.304 | 8690.420 |
| 10 | AciI-AciI | 438-477 | 40 | 12131.975 | 12612.049 |
| 11 | AciI-HaeIII | 478-497 | 20 | 6309.041 | 5463.877 |
| 12 | HaeIII-AciI | 498-593 | 96 | 29602.802 | 30349.930 |
| 13 | AciI-AciI | 594-595 | 2 | 636.108 | 636.108 |
| 14 | AciI-HaeIII | 596-598 | 3 | 965.160 | 307.056 |
| 15 | HaeIII-AciI | 599-676 | 78 | 23682.810 | 25155.101 |
| 16 | AciI-AciI | 677-703 | 27 | 8338.351 | 8378.358 |
| 17 | AciI-HaeIII | 704-714 | 11 | 3482.552 | 2731.464 |
| 18 | HaeIII-AciI | 715-875 | 161 | 49554.986 | 50556.215 |
| 19 | AciI-AciI | 876-923 | 48 | 14785.380 | 14901.439 |
| 20 | AciI-HaeIII | 924-928 | 5 | 1623.264 | 885.147 |
| 21 | HaeIII-HaeIII | 929-997 | 69 | 21418.494 | 21244.406 |
| 22 | HaeIII-HaeIII | 998-1073 | 76 | 23106.746 | 23875.875 |
| 23 | HaeIII-HaeIII | 1074-1095 | 22 | 6822.121 | 6804.097 |
| 24 | HaeIII-HaeIII | 1096-1151 | 56 | 17211.779 | 17420.821 |
| 25 | HaeIII-HaeIII | 1152-1183 | 32 | 10069.651 | 9731.578 |
| 26 | HaeIII-AciI | 1184-1217 | 34 | 10414.689 | 11241.830 |
| 27 | AciI-HaeIII | 1218-1247 | 30 | 9225.482 | 8723.443 |
| 28 | HaeIII-HaeIII | 1248-1277 | 30 | 9219.494 | 9348.524 |
| 29 | HaeIII-AciI | 1278-1292 | 15 | 4607.741 | 5025.817 |
| 30 | AciI-AciI | 1293-1296 | 4 | 1294.212 | 1214.200 |
| 31 | AciI-AciI | 1297-1303 | 7 | 2200.361 | 2160.355 |
| 32 | AciI-HaeIII | 1304-1307 | 4 | 1294.212 | 596.102 |
| 33 | HaeIII-AciI | 1308-1319 | 12 | 3786.598 | 4280.717 |
| 34 | AciI-HaeIII | 1320-1322 | 3 | 965.160 | 307.056 |
| 35 | HaeIII-HaeIII | 1323-1337 | 15 | 4655.764 | 4646.752 |
| 36 | HaeIII-HaeIII | 1338-1390 | 53 | 16142.619 | 16631.705 |
| 37 | HaeIII-HaeIII | 1391-1419 | 29 | 8796.425 | 9156.481 |
| 38 | HaeIII-AciI | 1420-1436 | 17 | 5208.849 | 5946.966 |
| 39 | AciI-AciI | 1437-1482 | 46 | 14343.361 | 14111.243 |
| 40 | AciI-HaeIII | 1483-1519 | 37 | 11670.946 | 10602.676 |
| 41 | HaeIII-HaeIII | 1520-1615 | 96 | 29689.915 | 29651.685 |
| 42 | HaeIII-AciI | 1616-1620 | 5 | 1567.263 | 2176.350 |
| 43 | AciI-HaeIII | 1621-1642 | 22 | 7008.147 | 6002.958 |
| 44 | HaeIII-AciI | 1643-1665 | 23 | 7071.160 | 7791.254 |
| 45 | AciI-AciI | 1666-1671 | 6 | 1887.304 | 1856.309 |
| 46 | AciI-HaeIII | 1672-1687 | 16 | 4992.832 | 4307.693 |
| 47 | HaeIII-HaeIII | 1688-1703 | 16 | 5014.815 | 4903.808 |
| 48 | HaeIII-AciI | 1704-1704 | 1 | 307.056 | 965.160 |
| 49 | AciI-AciI | 1705-1738 | 34 | 10512.724 | 10525.696 |
| 50 | AciI-HaeIII | 1739-1768 | 30 | 9181.516 | 8767.409 |
| 51 | HaeIII-HaeIII | 1769-1774 | 6 | 1887.304 | 1856.309 |
| 52 | HaeIII-AciI | 1775-1842 | 68 | 20976.445 | 21682.475 |
| 53 | AciI-HaeIII | 1843-1913 | 71 | 22161.566 | 21121.423 |
| 54 | HaeIII-HaeIII | 1914-1924 | 11 | 3491.563 | 3340.550 |
| 55 | HaeIII-AciI | 1925-1933 | 9 | 2691.457 | 3522.558 |
| 56 | AciI-(RightEnd) | 1934-1950 | 17 | 5249.851 | 4671.759 |

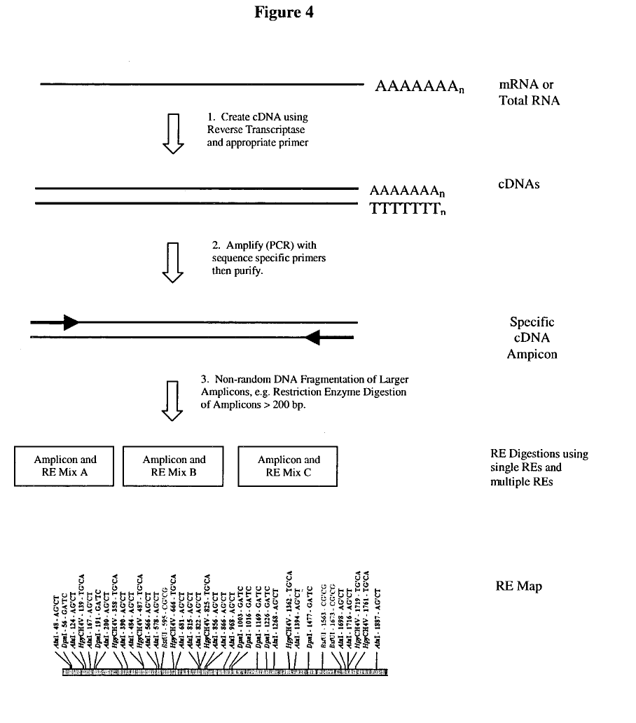

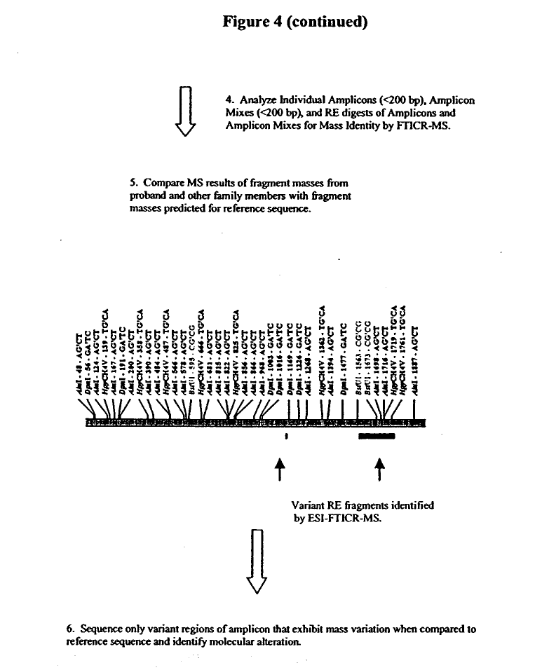

A schematic illustration of the method used to analyze the Pan1 and Pan2 cDNAs using ESI-FITCR is demonstrated in FIG. 4. Amplification of cDNAs performed herein may be omitted or modified as required. Fragmented Pan1 and Pan2 cDNAs are prepared and spectra are generated using ESI-FTICR-MS, which can be deconvoluted using standard deconvolution means, and compared to identify the region of Pan1 or Pan2 for each resulting fragment mass. FIG. 5a shows aligned partial spectra over the M/Z range from 952.5 to 957.5 for restriction enzyme digests of Pan1 and Pan2 cDNAs. Within the upper spectrum (Pan2), a unique molecular ion exists, (M-22H⁺⁾²²⁻, at a M/Z of 953.475. Deconvolution and analysis of this portion of the aligned spectra, shown in FIG. 5b, lowers the background and simplifies the pattern. Furthermore, at a M/Z ratio of 20,976.506 for the molecular ion (M-H⁺⁾¹⁻, the monoisotopic molecular weight is measured to be 20,976.506 daltons. Using Tables 1 and 2, which contain all of the fragments and their expected monoisotopic masses for Pan1 and Pan2 cDNAs, it is apparent that there is only a single fragment, the plus strand of fragment number 52 of the Pan2 digest, whose calculated mass matches that measured in FIG. 5b. Furthermore, the difference in the mass identity between the measured and the calculated is approximately 0.2 daltons (10 ppm), which would readily discriminate even a single nucleotide change, e.g. A to T transversion (9 daltons), within the same fragment.

FIG. 6a shows aligned partial spectra over the M/Z range from 1017.5 to 1027.0 for RE digests of Pan1 and Pan2 cDNAs. Within the upper spectrum (Pan2), a unique molecular ion exists, (M-29H⁺⁾²⁹⁻, at a M/Z of 1023.790. Deconvolution and analysis of this portion of the aligned spectra, shown in FIG. 6b, lowers the background and simplifies the pattern. Furthermore, at a M/Z ratio of 29,689.915 for the molecular ion (M-H⁺⁾¹⁻, the monoisotopic molecular weight is measured to be 29,689.929 daltons. Using Tables 1 and 2, which contain all of the double digestion fragments and their expected monoisotopic masses for Pan1 and Pan2 cDNAs, it is apparent that there is only a single fragment, the plus strand of fragment number 41 of the Pan2 digest, whose calculated mass matches that measured in FIG. 5b. Furthermore, the difference in the mass identity between the measured and the calculated is approximately 0.2 daltons (˜10 ppm), which would readily discriminate even a single nucleotide change, e.g. A to T transversion (9 daltons), within the same fragment.

Furthermore, the mass variants identified in FIGS. 5 and 6 overlap with the junctions that define the most dissimilar segment between Pan1 and Pan2 cDNA, basepairs 1576-1780 using the Pan1 coordinates. Accordingly, all of the double digested fragments between number 41 and 52 of Pan2 will differ in mass from those in Pan1.

### EXAMPLE 2

### Sequencing of Known Disease Genes for Medical Decision Making

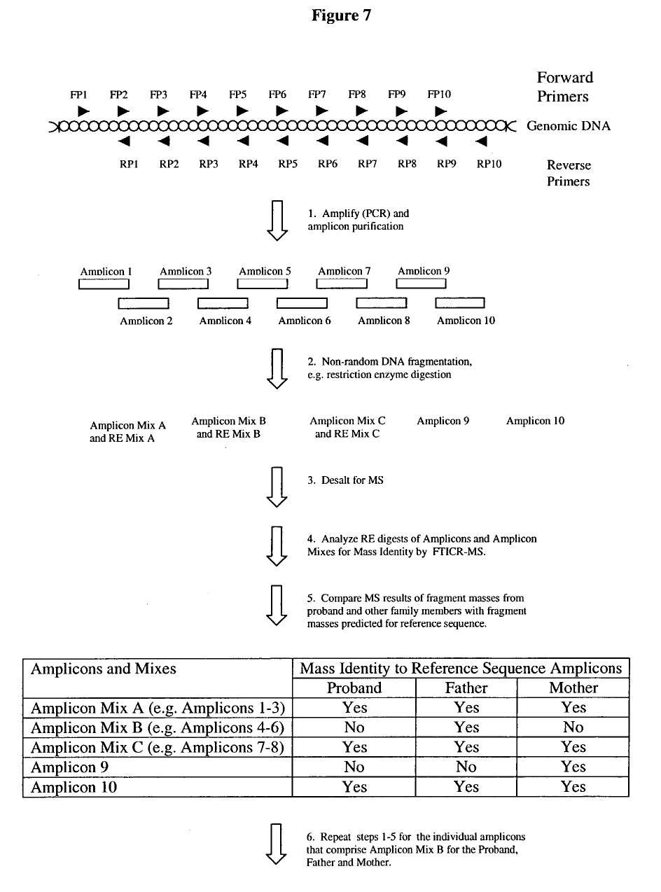

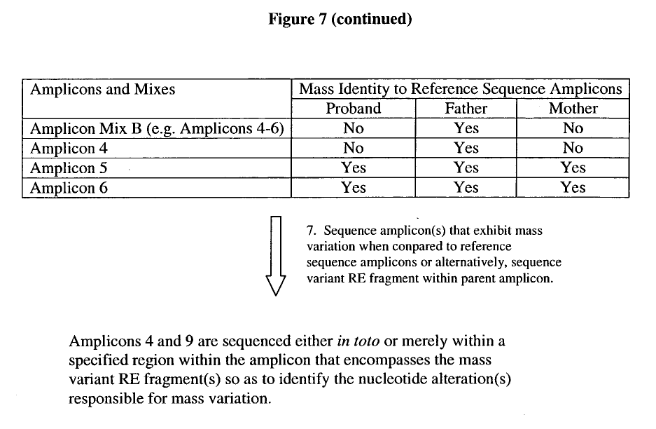

The following example demonstrates a method of the invention detecting polymorphisms in the CFTR gene using mass variation identification. The present invention allows the analysis of an entire gene for mass variation. The gene may be associated with a specific disease, such as the human cystic fibrosis transmembrane receptor (CFTR) gene. Alternatively, the gene may be analyzed for the presence of single nucleotide polymorphisms (SNPs) in nucleic acids derived from a subject (test nucleic acid or test DNA) or population of subjects. DNA fragments derived from a minimally tiled set of overlapping amplicons are derived by PCR of human genomic DNA. These amplicons may be of any size suitable for overlapping analysis, such as about 500 bases, 1 kb, 2 kb or greater. The exon organization of the CFTR gene is presented in Table 3. Exon lengths greater than 150 bases are indicated in bold in Table 3. A set of minimally overlapping amplicons is designed such that when amplified by PCR from genomic DNA, the complete gene is available for sequence validation based on mass analysis. Each amplicon will encode one or more introns and one or more exons. Primers can be positioned in either introns or exons but will preferably be positioned in unique, non-repetitive sequence stretches within introns. A schematic illustration of the method described in this example is provided in FIG. 7. FIG. 7 demonstrates the detectable changes in restriction enzyme fragment length of two mutations in the CFTR gene within amplicon 4 and amplicon 9. Table 4 provides the approximate location of forward and reverse primers and the exons that are included within the analysis such as to generate a tiling set of ˜2 kb amplicons. Amplicons are generated by PCR using a high fidelity, thermostable DNA polymerase or fragments thereof (Klenow-like), e.g. PfuI DNA polymerase, which lack both non-templated nucleotide polymerization activity and 3′ exonuclease activity.

TABLE 3
CFTR Gene Exon Organization

| | | |
|---|---|---|
| Gene | Coding | mRNA |

| Exon | Exon | Exon | Exon | Exon | Exon |
|---|---|---|---|---|---|
| Number | Start | End | Length | Start | End |
| 1a | −132 | 0 | 0 | 1 | 132 |
| 1b | 1 | 53 | 53 | 133 | 185 |
| 2 | 1000 | 1110 | 111 | 186 | 296 |
| 3 | 1564 | 1672 | 109 | 297 | 405 |
| 4 | 2086 | 2301 | 216 | 406 | 621 |
| 5 | 2750 | 2839 | 90 | 622 | 711 |
| 6a | 3393 | 3556 | 164 | 712 | 875 |
| 6b | 4689 | 4814 | 126 | 876 | 1001 |
| 7 | 5425 | 5671 | 247 | 1002 | 1248 |
| 8 | 6273 | 6365 | 93 | 1249 | 1341 |
| 9 | 7123 | 7305 | 183 | 1342 | 1524 |
| 10 | 8026 | 8217 | 192 | 1525 | 1716 |
| 11 | 8844 | 8938 | 95 | 1717 | 1811 |
| 12 | 9447 | 9533 | 87 | 1812 | 1898 |
| 13 | 10016 | 10739 | 724 | 1899 | 2622 |
| 14a | 11401 | 11529 | 129 | 2623 | 2751 |
| 14b | 12006 | 12043 | 38 | 2752 | 2789 |
| 15 | 12770 | 13020 | 251 | 2790 | 3040 |
| 16 | 13460 | 13539 | 80 | 3041 | 3120 |
| 17a | 14048 | 14198 | 151 | 3121 | 3271 |
| 17b | 14628 | 14855 | 228 | 3272 | 3499 |
| 18 | 15665 | 15765 | 101 | 3500 | 3600 |
| 19 | 16255 | 16503 | 249 | 3601 | 3849 |
| 20 | 16965 | 17120 | 156 | 3850 | 4005 |
| 21 | 17597 | 17686 | 90 | 4006 | 4095 |
| 22 | 18555 | 18727 | 173 | 4096 | 4268 |
| 23 | 19218 | 19323 | 106 | 4269 | 4374 |
| 24 | 20102 | 22018 | 198 | 4375 | 4572 |

TABLE 4
Amplicon Tiling Set to Amplify the CFTR Gene.

| Amplicon | Forward | Reverse | |
|---|---|---|---|
| Number | Primer | Primer | Exons Included |
| 1 | −50 | ˜2050 | 1a, 1b, 2 and 3 |
| 2 | ˜2010 | ˜4010 | 4, 5 and 6a |
| 3 | ˜3970 | ˜5970 | 6b and 7 |
| 4 | ˜5930 | ˜7930 | 8 and 9 |
| 5 | ˜7890 | ˜9890 | 10, 11 and 12 |
| 6 | ˜9850 | ˜11850 | 13 and 14a |
| 7 | ˜11810 | ˜13810 | 14b, 15 and 16 |
| 8 | ˜13780 | ˜15880 | 17a, 17b and 18 |
| 9 | ˜18840 | ˜17840 | 19, 20 and 21 |
| 10 | ˜17800 | ˜20350 | 22, 23 and 24* |

*only the coding region of exon 24 is included in Amplicon 10.

Multiple amplicons can be generated simultaneously as part of one or more multiplex PCR reactions. Alternatively, amplicons can be generated individually and then optionally mixed with other amplicons in a predetermined manner prior to DNA fragmentation.

The amplicons will be fragmented using one or more sequence specific DNA hydrolases, e.g. restriction enzymes, universal enzymes, etc., whose recognition site is small and therefore occurs frequently in double stranded DNA. Based on the frequency of occurrence of restriction enzyme sites within a designated amplicon, amplicons are digested using one or more restriction enzymes to cleave the DNA such that the resulting fragments are less than, e.g., 100 bp in length. The amplicons are singly digested, or alternatively, mixed in different combinations such that mix 1, comprised of two or more amplicons, is digested with a unique combination of restriction enzymes (REs), e.g., RE 1-3, and mix 2, also comprised of two or more amplicons, is digested with a combination of REs, e.g. RE 1, 3, and 4. Additional amplicon mixes are assembled and digested appropriately to generate restriction enzyme fragments that can be unambiguously distinguished from other fragments within the digest by fragment mass determinations utilizing mass spectrometers (MS), preferably utilizing ESI-FTICR, that determine M/Z with high range, resolution, and accuracy e.g. ≦200 bp, 30,000 and >0.01%, respectively.

### EXAMPLE 3

### Detection of Polymorphisms in Coding Regions and Splice Junctions of Disease-Causing Genes

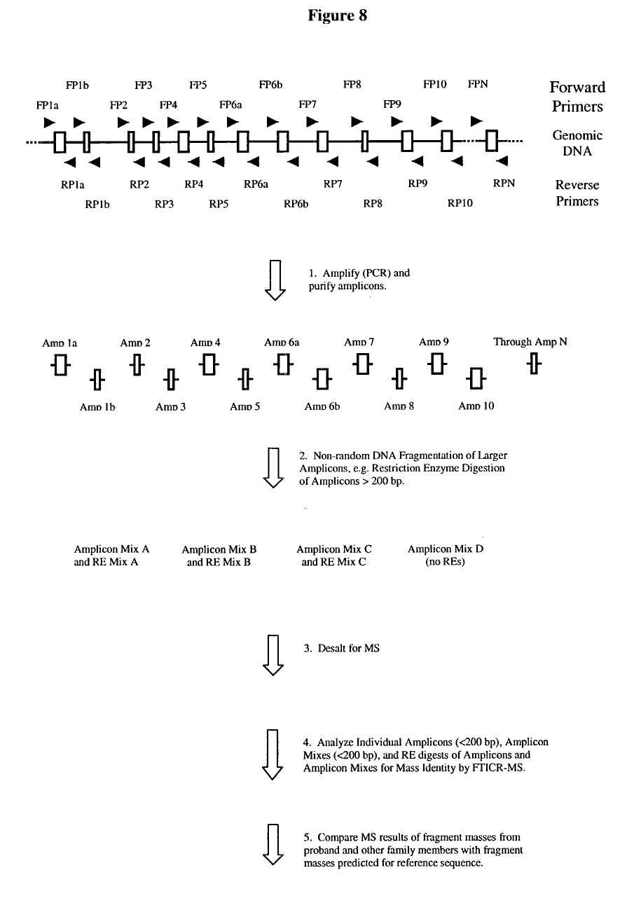

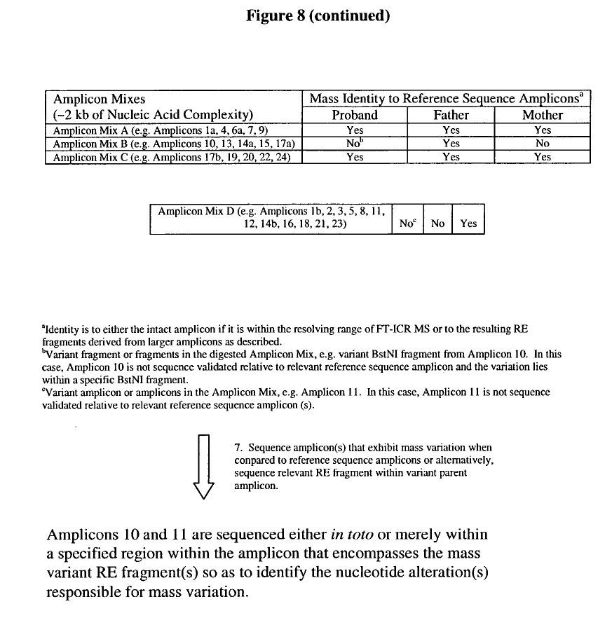

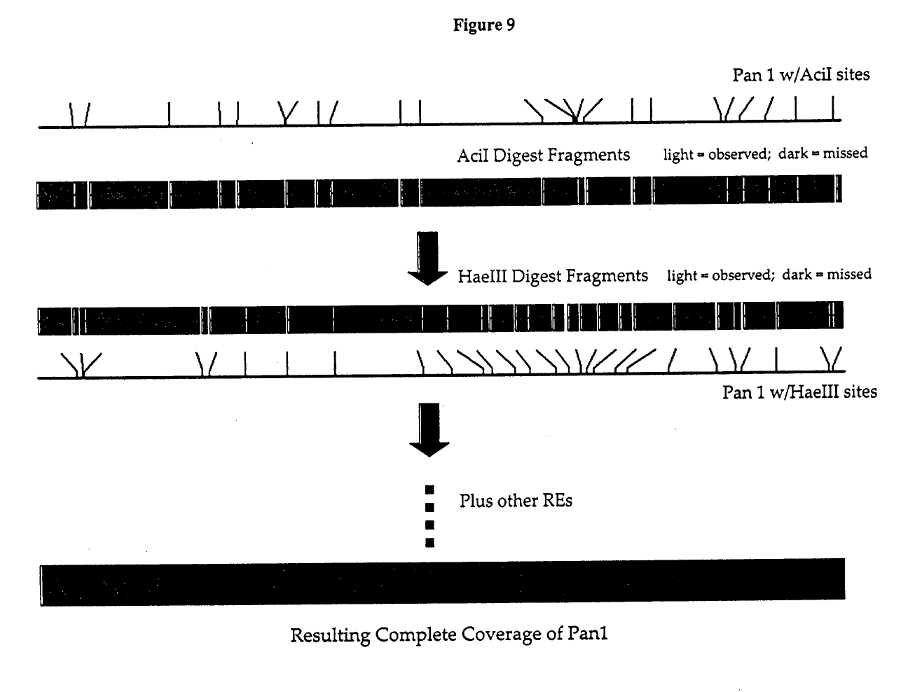

The following example demonstrates the methods of the invention applied to detection of polymorphisms in the CFTR coding and splice regions using mass variation identification. The present invention allows the detection of putative mutations, variants or polymorphisms within a gene of interest such as the CFTR gene, and can be focused towards the exons and proximal intron regions encoding splice junctions. Using the exon organization provided above in Table 3, a set of non-overlapping amplicons are designed such that when amplified by PCR from genomic DNA, the entirety of the exons and their respective proximal introns junctions are available for sequence validation and polymorphism based on mass analysis. Each amplicon encodes a single exon and proximal segments of both upstream and downstream flanking introns. The forward primer is positioned in the upstream intron and the reverse primer is positioned in the downstream intron relative to the exon to be amplified. All primers are preferably positioned in unique, non-repetitive sequence stretches within introns and anneal to their respective complementary strand at similar thermodynamic stability to enable amplification conditions to be uniform for all amplicons. A schematic illustration of the method described in this example is provided in FIG. 8. Table 5 provides the approximate location of forward and reverse primers for each amplicon, the exon that is included within the respective amplicon, and the size of the resulting amplicon. Amplicons are generated by PCR using a high fidelity, thermostable DNA polymerase or fragments thereof (Klenow-like), e.g. PfuI DNA polymerase, which lack both non-templated nucleotide polymerization activity and 3′ exonuclease activity. Multiple amplicons are generated simultaneously as part of one or more multiplex PCR reactions. Alternatively, amplicons are generated individually and then optionally mixed with other amplicons in a predetermined manner for DNA fragmentation.

TABLE 5
Amplicon Set for All Exons and Proximal Segments
of Flanking Introns of the CFTR Gene

| Amplicon | | | |
|---|---|---|---|
| (Exon) | Forward | Reverse | Amplicon |
| Number | Primer | Primer | Size (bp) |
| 1a | −172 | 40 | 212 |
| 1b | −40 | 93 | 133 |
| 2 | 960 | 1150 | 190 |
| 3 | 1524 | 1712 | 188 |
| 4 | 2046 | 2341 | 295 |
| 5 | 2710 | 2879 | 169 |
| 6a | 3353 | 3596 | 243 |
| 6b | 4649 | 4854 | 205 |
| 7 | 5385 | 5711 | 326 |
| 8 | 6233 | 6405 | 172 |
| 9 | 7083 | 7345 | 262 |
| 10 | 7986 | 8257 | 271 |
| 11 | 8804 | 8978 | 174 |
| 12 | 9407 | 9573 | 166 |
| 13 | 9976 | 10779 | 803 |
| 14a | 11361 | 11569 | 208 |
| 14b | 11966 | 12083 | 117 |
| 15 | 12730 | 13060 | 330 |
| 16 | 13420 | 13579 | 159 |
| 17a | 14008 | 14238 | 230 |
| 17b | 14588 | 14895 | 307 |
| 18 | 15625 | 15805 | 180 |
| 19 | 16215 | 16543 | 328 |
| 20 | 16925 | 17160 | 235 |
| 21 | 17557 | 17726 | 169 |
| 22 | 18515 | 18767 | 252 |
| 23 | 19178 | 19363 | 185 |
| 24 | 20062 | 20300 | 238 |

In Table 5, the entries under “amplicon size” assumes 20 nt length forward and reverse primers and an additional 20 residue spacer between the 3′ end of each primer and the exon portion of the amplicon. Consequently, each amplicon is ˜80 bp greater than the size of the exon. Amplicons of greater or lesser size can be generated by re-positioning the forward and or reverse primers into neighboring single-copy regions of appropriate thermodynamic stability. Amplicons depicted in bold have a size greater than 200 bp, which may require fragmentation prior to MS analysis.

Table 6 demonstrates the detectable changes in restriction enzyme fragment length of two mutations in exon 10 the CFTR gene. The CFTR exon 10 can be amplified to generate a 210 basepair amplicon. The delta 508 mutation of CFTR exon 10 results in a 207 basepair amplicon, and the delta 507 mutation of CFTR exon 10 results in a 207 basepair amplicon. The altercations in restriction enzyme fragment length can be observed when the CFTR exon 10 amplicon is digested with a single restriction enzyme or two restriction enzymes. Masses differing between wild-type CFTR exon 10 and the delta 508 and the delta 507 mutations are indicated in bold. For example, digestion of the wild-type amplicon with BstNI generates a restriction enzyme fragment that is 79 bases in length from the 3′most BstNI site to the 3′ end of the amplicon (plus strand) with a monoisotopic mass of 24439.051 Da, while the corresponding restriction enzyme fragment resulting from digestion of either the delta 508 and delta 507 mutant amplicons with BstNI is 76 bases in length (plus strand) with a monoisotopic mass of 23526.914 Da, a 3 base decrease that results in a decrease in mass of 912.137 Da.

TABLE 6

| | | Strand Length | | | Strand Mass (monoisotopic) | | |
|---|---|---|---|---|---|---|---|
| Termini | Strand | wt | Δ508 | Δ507 | wt | Δ508 | Δ507 |

BstNl (CC′WGG) cuts at 120 and 131 bp generating
fragments of 120, 11 and 79

| | | | | | | | |
|---|---|---|---|---|---|---|---|
| Left-BstNI | plus | 120 | 120 | 120 | 37135.056 | 37135.056 | 37135.056 |
| | minus | 121 | 121 | 121 | 37311.164 | 37311.164 | 37311.164 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNI-Right | plus | 79 | 76 | 76 | 24439.051 | 23526.914 | 23532.902 |
| | minus | 78 | 75 | 75 | 24062.913 | 23123.741 | 23116.758 |

MseI (T′TAA) cuts at 80 and 140 generating
fragments of 80, 60 and 70

| | | | | | | | |
|---|---|---|---|---|---|---|---|
| Left-MseI | plus | 80 | 80 | 80 | 24828.064 | 24828.064 | 24828.064 |
| | minus | 82 | 82 | 82 | 25223.153 | 25223.153 | 25223.153 |
| MseI-MseI | plus | 60 | 60 | 60 | 18491.996 | 18491.996 | 18491.996 |
| | minus | 60 | 60 | 60 | 18595.083 | 18595.083 | 18595.083 |
| MseI-Right | plus | 70 | 67 | 67 | 21679.603 | 20767.466 | 20773.454 |
| | minus | 68 | 65 | 65 | 20959.413 | 20020.241 | 20013.257 |

NIaIV (GGN′NCC) cuts at 62 and 135 generating
fragments of 62, 73 and 75

| | | | | | | | |
|---|---|---|---|---|---|---|---|
| Left-NIaIV | plus | 62 | 62 | 62 | 19221.139 | 19221.139 | 19221.139 |
| | minus | 62 | 62 | 62 | 19097.161 | 19097.161 | 19097.161 |
| NIaIV | plus | 73 | 73 | 73 | 22590.669 | 22590.669 | 22590.669 |
| | minus | 73 | 73 | 73 | 22524.720 | 22524.720 | 22524.720 |
| NIaIV-Right | plus | 75 | 72 | 72 | 23187.855 | 22275.718 | 22281.706 |
| | minus | 75 | 72 | 72 | 23155.769 | 22216.597 | 22209.613 |

Tsp509I (″AATT) cuts at 77 and 95 generating
fragments of 77, 18 and 115

| | | | | | | | |
|---|---|---|---|---|---|---|---|
| Left-Tsp509I | plus | 77 | 77 | 77 | 23897.904 | 23897.904 | 23897.904 |
| | minus | 81 | 81 | 81 | 24919.108 | 24919.108 | 24919.108 |
| Tsp509I- | | | | | | | |
| Tsp509I | plus | 18 | 18 | 18 | 5657.958 | 5657.958 | 5657.958 |
| | minus | 18 | 18 | 18 | 5492.881 | 5492.881 | 5492.881 |
| Tsp509I-Right | plus | 115 | 112 | 112 | 35443.801 | 34531.664 | 34537.652 |
| | minus | 111 | 108 | 108 | 34365.660 | 33426.488 | 33419.505 |

BstNI (CC′WGG) and MseI (TTAA) cut at 80, 120, 131 and
140 bp generating fragments of 80, 40, 11, 9 and 70

| | | | | | | | |
|---|---|---|---|---|---|---|---|
| Left-MseI | plus | 80 | 80 | 80 | 24828.064 | 24828.064 | 24828.064 |
| | minus | 82 | 82 | 82 | 25223.153 | 25223.153 | 25223.153 |
| MseI-BstNI | plus | 40 | 40 | 40 | 12325.001 | 12325.001 | 12325.001 |
| | minus | 39 | 39 | 39 | 12106.020 | 12106.020 | 12106.020 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNI-MseI | plus | 9 | 9 | 9 | 2777.458 | 2777.458 | 2777.458 |
| | minus | 10 | 10 | 10 | 3121.510 | 3121.510 | 3121.510 |
| MseI-Right | plus | 70 | 67 | 67 | 21679.603 | 20767.466 | 20773.454 |
| | minus | 68 | 65 | 65 | 20959.413 | 20020.241 | 20013.257 |

BstNI (CC′WGG) and NIaIV (GGN′NCC) cut at 62, 120, 131, and
135 bp generating fragments of 62, 58, 11, 4, and 75.

| | | | | | | | |
|---|---|---|---|---|---|---|---|
| Left-NIaIV | plus | 62 | 62 | 62 | 19221.139 | 19221.139 | 19221.139 |
| | minus | 62 | 62 | 62 | 19097.161 | 19097.161 | 19097.161 |
| NIaIV-BstNI | plus | 58 | 58 | 58 | 17931.927 | 17931.927 | 17931.927 |
| | minus | 59 | 59 | 59 | 18232.013 | 18232.013 | 18232.013 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNl-NIaIV | plus | 4 | 4 | 4 | 1269.206 | 1269.206 | 1269.206 |
| | minus | 3 | 3 | 3 | 925.154 | 925.154 | 925.154 |
| NIaIV-Right | plus | 75 | 72 | 72 | 23187.855 | 22275.718 | 22281.706 |
| | minus | 75 | 72 | 72 | 23155.769 | 22216.597 | 22209.613 |

BstNI (CC′WGG) and Tsp509I (′AATT) cut at 77, 95, 120, and
131 bp generating fragments of 77, 18, 25, 11, and 79 bp.

| | | | | | | | |
|---|---|---|---|---|---|---|---|
| Left-TSp509I | plus | 77 | 77 | 77 | 23897.904 | 23897.904 | 23897.904 |
| | minus | 81 | 81 | 81 | 24919.108 | 24919.108 | 24919.108 |
| Tsp509I- | | | | | | | |
| Tsp509I | plus | 18 | 18 | 18 | 5657.958 | 5657.958 | 5657.958 |
| | minus | 18 | 18 | 18 | 5492.881 | 5492.881 | 5492.881 |
| Tsp509I-BstNI | plus | 25 | 25 | 25 | 7615.213 | 7615.213 | 7615.213 |
| | minus | 22 | 22 | 22 | 6935.194 | 6935.194 | 6935.194 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNI-Right | plus | 79 | 76 | 76 | 24439.051 | 23526.914 | 23532.902 |
| | minus | 78 | 75 | 75 | 24062.913 | 23123.741 | 23116.758 |

MseI (T′TAA) and NIaIV (GGN′NCC) cut at 62, 80, 135 and 140 bp
generating fragments of 62, 18, 55, 5, and 70 bp.

| | | | | | | | |
|---|---|---|---|---|---|---|---|
| Left-NIaIV | plus | 62 | 62 | 62 | 19221.139 | 19221.139 | 19221.139 |
| | minus | 62 | 62 | 62 | 19097.161 | 19097.161 | 19097.161 |
| NIaIV-MseI | plus | 18 | 18 | 18 | 5624.935 | 5624.935 | 5624.935 |
| | minus | 20 | 20 | 20 | 6144.002 | 6144.002 | 6144.002 |
| MseI-NIaIV | plus | 55 | 55 | 55 | 16983.744 | 16983.744 | 16983.744 |
| | minus | 53 | 53 | 53 | 16398.727 | 16398.727 | 16398.727 |
| NIaIV-MseI | plus | 5 | 5 | 5 | 1526.262 | 1526.262 | 1526.262 |
| | minus | 7 | 7 | 7 | 2214.300 | 2214.300 | 2214.300 |
| MseI-Right | plus | 70 | 67 | 67 | 21679.603 | 20767.466 | 20773.454 |
| | minus | 68 | 65 | 65 | 20959.413 | 20020.241 | 20013.257 |

MseI (T′TAA) and Tsp509I (″AATT) cuts at cut at 77, 80, 95 and
140 bp generating fragments of 77, 3, 15, 45, and 70 bp.

| | | | | | | | |
|---|---|---|---|---|---|---|---|
| Left-Tsp509I | plus | 77 | 77 | 77 | 23897.904 | 23897.904 | 23897.904 |
| | minus | 81 | 81 | 81 | 24919.108 | 24919.108 | 24919.108 |
| Tsp509I-MseI | plus | 3 | 3 | 3 | 948.170 | 948.170 | 948.170 |
| | minus | 1 | 1 | 1 | 322.055 | 322.055 | 322.055 |
| MseI-Tsp509I | plus | 15 | 15 | 15 | 4727.798 | 4727.798 | 4727.798 |
| | minus | 17 | 17 | 17 | 5188.836 | 5188.836 | 5188.836 |
| Tsp509I-MseI | plus | 45 | 45 | 45 | 13782.208 | 13782.208 | 13782.208 |
| | minus | 43 | 43 | 43 | 13424.257 | 13424.257 | 13424.257 |
| MseI-Right | plus | 70 | 67 | 67 | 21679.603 | 20767.466 | 20773.454 |
| | minus | 68 | 65 | 65 | 20959.413 | 20020.241 | 20013.257 |

NIaIV (GGN′NCC) and Tsp509I (′AATT) cut at 62, 77, 95 and 135 bp
generating fragments of 62, 15, 18, 40, and 135 bp.

| | | | | | | | |
|---|---|---|---|---|---|---|---|
| Left-NIaIV | plus | 62 | 62 | 62 | 19221.139 | 19221.139 | 19221.139 |
| | minus | 62 | 62 | 62 | 19097.161 | 19097.161 | 19097.161 |
| NIaIV-Tsp509I | plus | 15 | 15 | 15 | 4694.775 | 4694.775 | 4694.775 |
| | minus | 19 | 19 | 19 | 5839.957 | 5839.957 | 5839.957 |
| Tsp509I- | | | | | | | |
| Tsp509I | plus | 18 | 18 | 18 | 5657.958 | 5657.958 | 5657.958 |
| | minus | 18 | 18 | 18 | 5492.881 | 5492.881 | 5492.881 |
| Tsp509I-NIaIV | plus | 40 | 40 | 40 | 12273.955 | 12273.955 | 12273.955 |
| | minus | 36 | 36 | 36 | 11227.901 | 11227.901 | 11227.901 |
| NIaIV-Right | plus | 75 | 72 | 72 | 23187.855 | 22275.718 | 22281.706 |
| | minus | 75 | 72 | 72 | 23155.769 | 22216.597 | 22209.613 |

CFTR amplicons whose size is within the resolving range of Fr-ICR are analyzed for mass variation without fragmentation. These amplicons will be examined for mass variation either individually or as mixtures with other amplicons that are also within the resolving range of the FT-ICR.

Amplicons whose size is beyond the resolving range of FT-ICR are fragmented prior to analysis for mass variation, as described supra. Based on the frequency of occurrence of restriction enzyme sites within a designated amplicon, amplicons are digested using one or more restriction enzymes to cleave the DNA such that the resulting fragments are less than, e.g., about 100 bp in length. The amplicons are singly digested or, alternatively, mixed in different combinations such that mix 1, comprised of two or more amplicons, is digested with a combination of restriction enzymes, e.g. RE 1-3. Then, mix 2, also comprised of two or more amplicons, is digested with a combination of restriction enzymes, e.g. RE 1, 3, and 4. Additional amplicon mixes are assembled and digested appropriately to generate RE fragments whose sizes are within the range of resolution by mass spectrometry and can be unambiguously distinguished from other fragments within the digest by fragment mass determinations utilizing mass spectrotrometers (MS), preferably utilizing ESI-FTICR. Mass spectrometers such as these are able to determine M/Z with high range, resolution, and accuracy e.g. <200 bp, 30,000 and >0.01%, respectively.

To analyze Mendelian inheritance of genetic diseases or disease predispositions, it is beneficial to have access to genomic DNA from the parents, siblings, and other first-degree relatives in addition to the test subject (the proband). Accordingly, amplification of the exons and splice regions of the CFTR gene is performed for each member in the family for which genomic DNA is available. Once amplified, each set of amplicons for individual family members are fragmented, analyzed by ESI-FTICR and then compared to a reference set of amplicons derived from genomic DNA of known sequence, or alternatively, compared to a database containing masses of predicted amplicons. Mass analyses that reveal differences between one or more amplicons (and resulting RE fragments) derived from test DNAs and the appropriate reference set of amplicons (and resulting RE fragments) will denote variant amplicons that encode a sequence different than that of the reference sequence. Furthermore, variant and invariant amplicons derived from the test subject (proband) should be consistent with Mendelian inheritance. Exceptions to this prediction may arise due to somatic mutations within the discordant amplicon. When mass variant amplicon mixes are identified, the mass analysis determination is repeated with individual amplicons that comprised the original amplicon mix to ascertain which amplicon or amplicons show mass variation. After indentifying individual amplicons that fail to validate the reference sequence, those amplicons will be sequenced either completely or within intervals that will encompass restriction enzyme fragments of variant mass when compared to the standards predicted by the reference sequence.

### EXAMPLE 4

### Detection of Polymorphisms in Coding Regions and Splice Junctions of Disease-Causing Genes

The following example further explores the experiments described in Example 3 to apply the methods of the present invention to the detection of polymorphisms in the CFTR coding and splice regions using mass variation identification. Using the exon organization provided above in Table 3, a set of non-overlapping amplicons are designed as described in Example 3. Table 7 provides the approximate location of forward and reverse primers for each amplicon, the exon that is included within the respective amplicon, and the size of the resulting amplicon.

TABLE 7
Amplicon Set for All Exons and Proximal Segments
of Flanking Introns of the CFTR Gene

| Amplicon | | | |
|---|---|---|---|
| (Exon) | Forward | Reverse | Amplicon |
| Number | Primer | Primer | Size (bp) |
| 1a | −172 | 40 | 212 |
| 1b | −40 | 93 | 133 |
| 2 | 960 | 1150 | 190 |
| 3 | 1524 | 1712 | 188 |
| 4 | 2046 | 2341 | 295 |
| 5 | 2710 | 2879 | 169 |
| 6a | 3353 | 3596 | 243 |
| 6b | 4649 | 4854 | 205 |
| 7 | 5385 | 5711 | 326 |
| 8 | 6233 | 6405 | 172 |
| 9 | 7083 | 7345 | 262 |
| 10 | 7986 | 8257 | 271 |
| 11 | 8804 | 8978 | 174 |
| 12 | 9407 | 9573 | 166 |
| 13 | 9976 | 10779 | 803 |
| 14a | 11361 | 11569 | 208 |
| 14b | 11966 | 12083 | 117 |
| 15 | 12730 | 13060 | 330 |
| 16 | 13420 | 13579 | 159 |
| 17a | 14008 | 14238 | 230 |
| 17b | 14588 | 14895 | 307 |
| 18 | 15625 | 15805 | 180 |
| 19 | 16215 | 16543 | 328 |
| 20 | 16925 | 17160 | 235 |
| 21 | 17557 | 17726 | 169 |
| 22 | 18515 | 18767 | 252 |
| 23 | 19178 | 19363 | 185 |
| 24 | 20062 | 20300 | 238 |

In Table 7, the entries under “amplicon size” assumes 20 nt length forward and reverse primers and an additional 20 residue spacer between the 3′ end of each primer and the exon portion of the amplicon. Consequently, each amplicon is ˜80 bp greater than the size of the exon. Amplicons of greater or lesser size can be generated by re-positioning the forward and or reverse primers into neighboring single-copy regions of appropriate thermodynamic stability. Amplicons depicted in bold have a size greater than 200 bp, which may require fragmentation prior to MS analysis.

Table 8 demonstrates the detectable changes in restriction enzyme fragment length of two mutations in exon 10 the CFTR gene. Using a primer selection program to design the primers for amplification, the CFTR exon 10 is amplified to generate a 280 basepair amplicon. The delta 508 mutation of CFTR exon 10 results in a change at nucleotides 184-186, and the delta 507 mutation of CFTR exon 10 results in a change at nucleotides 181-184. The alteration in restriction enzyme fragment length can be observed when the CFTR exon 10 amplicon is digested with a single restriction enzyme or two restriction enzymes. For example, digestion of the wild-type amplicon with BstNI generates a restriction enzyme fragment is 122 bases in length from the 3′most BstNI site to the 3′ end of the amplicon (plus strand), while the corresponding restriction enzyme fragment resulting from digestion of either the delta 508 and delta 507 mutant amplicons with BstNI is 119 bases in length (plus strand), a 3 base decrease that can be detected by the mass spectrometric methods of the present invention.

TABLE 8

| | | Strand Length | | | Strand Mass (monoisotopic) | | |
|---|---|---|---|---|---|---|---|
| Termini | Strand | wt | Δ508 | Δ507 | wt | Δ508 | Δ507 |

BstNI (CC′WGG) cuts at 147 and 158 bp generating
fragments of 147, 11 and 122 in wt

| | | | | | | | |
|---|---|---|---|---|---|---|---|
| Left-BstNI | plus | 147 | 147 | 147 | 45546.430 | 45546.430 | 45546.430 |
| | minus | 148 | 148 | 148 | 45571.524 | 45571.524 | 45571.524 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNI-Right | plus | 122 | 119 | 119 | 37831.273 | 36919.136 | 36925.124 |
| | minus | 121 | 118 | 118 | 37219.057 | 36279.886 | 36272.902 |

MseI (T′TAA) cuts at 107 and 167 generating
fragments of 107, 60 and 113 in wt.

| | | | | | | | |
|---|---|---|---|---|---|---|---|
| Left-MseI | plus | 107 | 107 | 107 | 33239.438 | 33239.438 | 33239.438 |
| | minus | 109 | 109 | 109 | 33483.513 | 33483.513 | 33483.513 |
| MseI-MseI | plus | 60 | 60 | 60 | 18491.996 | 18491.996 | 18491.996 |
| | minus | 60 | 60 | 60 | 18595.083 | 18595.083 | 18595.083 |
| MseI-Right | plus | 113 | 110 | 110 | 35071.825 | 34159.688 | 34165.676 |
| | minus | 111 | 108 | 108 | 34115.557 | 33176.385 | 33169.402 |

NlaIV (GGN′NCC) cuts at 89 and 162 generating
fragments of 89, 73 and 118 in wt.

| | | | | | | | |
|---|---|---|---|---|---|---|---|
| Left-NIaIV | plus | 89 | 89 | 89 | 27632.512 | 27632.512 | 27632.512 |
| | minus | 89 | 89 | 89 | 27357.520 | 27357.520 | 27357.520 |
| NIaIV | plus | 73 | 73 | 73 | 22590.669 | 22590.669 | 22590.669 |
| | minus | 73 | 73 | 73 | 22524.720 | 22524.720 | 22524.720 |
| NIaIV-Right | plus | 118 | 115 | 115 | 36580.077 | 35667.940 | 35673.928 |
| | minus | 118 | 115 | 115 | 36311.913 | 35372.741 | 35365.758 |

Tsp509I (″AATT) cuts at 104 and 122 generating
fragments of 104, 18 and 158 in wt.

| | | | | | | | |
|---|---|---|---|---|---|---|---|
| Left-Tsp509I | plus | 104 | 104 | 104 | 32309.277 | 32309.277 | 32309.277 |
| | minus | 108 | 108 | 108 | 33179.468 | 33179.468 | 33179.468 |
| Tsp509I-Tsp509I | plus | 18 | 18 | 18 | 5657.958 | 5657.958 | 5657.958 |
| | minus | 18 | 18 | 18 | 5492.881 | 5492.881 | 5492.881 |
| Tsp509I-Right | plus | 158 | 155 | 155 | 48836.023 | 47923.886 | 47929.874 |
| | minus | 154 | 151 | 151 | 47521.805 | 46582.633 | 46575.650 |

BstNI (CC′WGG) and MseI (T′TAA) cut at 107, 147, 158and 167 bp
generating fragments of 107, 40, 11, 9 and 113 in wt.

| | | | | | | | |
|---|---|---|---|---|---|---|---|
| Left-MseI | plus | 107 | 107 | 107 | 33239.438 | 33239.438 | 33239.438 |
| | minus | 109 | 109 | 109 | 33483.513 | 33483.513 | 33483.513 |
| MseI-BstNI | plus | 40 | 40 | 40 | 12325.001 | 12325.001 | 12325.001 |
| | minus | 39 | 39 | 39 | 12106.020 | 12106.020 | 12106.020 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNI-MseI | plus | 9 | 9 | 9 | 2777.458 | 2777.458 | 2777.458 |
| | minus | 10 | 10 | 10 | 3121.510 | 3121.510 | 3121.510 |
| MseI-Right | plus | 113 | 110 | 110 | 35071.825 | 34159.688 | 34165.676 |
| | minus | 111 | 108 | 108 | 34115.557 | 33176.385 | 33169.402 |

BstNI (CC′WGG) and NIaIV (GGN′NCC) cut at 89, 147, 158 and 162 bp
generating fragments of 89, 58, 11, 4, and 118 in wt.

| | | | | | | | |
|---|---|---|---|---|---|---|---|
| Left-NIaIV | plus | 89 | 89 | 89 | 27632.512 | 27632.512 | 27632.512 |
| | minus | 89 | 89 | 89 | 27357.520 | 27357.520 | 27357.520 |
| NIaIV-BstNI | plus | 58 | 58 | 58 | 17931.927 | 17931.927 | 17931.927 |
| | minus | 59 | 59 | 59 | 18232.013 | 18232.013 | 18232.013 |
| BstNI-BstNI | plus | 11 | 11 | 11 | 3425.556 | 3425.556 | 3425.556 |
| | minus | 11 | 11 | 11 | 3403.573 | 3403.573 | 3403.573 |
| BstNI-NIaIV | plus | 4 | 4 | 4 | 1269.206 | 1269.206 | 1269.206 |
| | minus | 3 | 3 | 3 | 925.154 | 925.154 | 925.154 |
| NIaIV-Right | plus | 118 | 115 | 115 | 36580.077 | 35667.940 | 35673.928 |
| | minus | 118 | 115 | 115 | 36311.913 | 35372.741 | 35365.758 |

BstNI (CC′WGG) and Tsp509I (′AATT) cut at 104, 122, 147, and 158 bp
generating fragments of 104, 18, 25, 11, and 122 bp in wt.

| | | | | | | | |
|---|---|---|---|---|---|---|---|
| Left-TSp509I | plus | 104 | 104 | 104 | 32309.277 | 32309.277 | 32309.277 |
| | minus | 108 | 108 | 108 | 33179.468 | 33179.468 | 33179.468 |
| Tsp509I-Tsp509I | plus | 18 | 18 | 18 | 5657.958 | 5657.958 | 5657.958 |
| | minus | 18 | 18 | 18 | 5492.881 | 5492.881 | 5492.881 |
| Tsp509I-BstNI | plus | 25 | 25 | 25 | 7615.213 | 7615.213 | 7615.213 |
| | minus | 22 | 22 | 22 | 6935.194 | 6935.194 | 6935.194 |
| BstNI-BstNI | plus | 11 | 11 | 11 | 3425.556 | ,3425.556 | 3425.556 |
| | minus | 11 | 11 | 11 | 3403.573 | 3403.573 | 3403.573 |
| BstNI-Right | plus | 122 | 119 | 119 | 37831.273 | 36919.136 | 36925.124 |
| | minus | 121 | 118 | 118 | 37219.057 | 36279.886 | 36272.902 |

MseI (TTAA) and NIaIV (GGN′NCC) cut at 89, 107, 162 and 167 bp
generating fragments of 89, 18, 55, 5, and 113 bp.

| | | | | | | | |
|---|---|---|---|---|---|---|---|
| Left-NIaIV | plus | 89 | 89 | 89 | 27632.512 | 27632.512 | 27632.512 |
| | minus | 89 | 89 | 89 | 23764.952 | 23764.952 | 23764.952 |
| NIaIV-MseI | plus | 18 | 18 | 18 | 5624.935 | 5624.935 | 5624.935 |
| | minus | 20 | 20 | 20 | 6144.002 | 6144.002 | 6144.002 |
| MseI-NIaIV | plus | 55 | 55 | 55 | 16983.744 | 16983.744 | 16983.744 |
| | minus | 53 | 53 | 53 | 16398.727 | 16398.727 | 16398.727 |
| NIaIV-MseI | plus | 5 | 5 | 5 | 1526.262 | 1526.262 | 1526.262 |
| | minus | 7 | 7 | 7 | 2214.300 | 2214.300 | 2214.300 |
| MseI-Right | plus | 113 | 110 | 110 | 35071.825 | 34159.688 | 34165.676 |
| | minus | 111 | 108 | 108 | 34115.557 | 33176.385 | 33169.402 |

MseI (T′TAA) and Tsp509I (″AATT) cuts at cut at 77, 80, 95 and 140 bp
generating fragments of 77, 3, 15, 45, and 70 bp in wt.

| | | | | | | | |
|---|---|---|---|---|---|---|---|
| Left-Tsp509I | plus | 77 | 77 | 77 | 32309.277 | 32309.277 | 32309.277 |
| | minus | 81 | 81 | 81 | 33179.468 | 33179.468 | 33179.468 |
| Tsp509I-MseI | plus | 3 | 3 | 3 | 948.170 | 948.170 | 948.170 |
| | minus | 1 | 1 | 1 | 322.055 | 322.055 | 322.055 |
| MseI-Tsp509I | plus | 15 | 15 | 15 | 4727.798 | 4727.798 | 4727.798 |
| | minus | 17 | 17 | 17 | 5188.836 | 5188.836 | 5188.836 |
| Tsp509I-MseI | plus | 45 | 45 | 45 | 13782.208 | 13782.208 | 13782.208 |
| | minus | 43 | 43 | 43 | 13424.257 | 13424.257 | 13424.257 |
| MseI-Right | plus | 70 | 67 | 67 | 35071.825 | 34159.688 | 34165.676 |
| | minus | 68 | 65 | 65 | 34115.557 | 33176.385 | 33169.402 |

NIaIV (GGN′NCC) and Tsp509I (′AATT) cut at 89, 104, 122 and 162 bp
generating fragments of 89, 15, 18, 40, and 118 bp in wt.

| | | | | | | | |
|---|---|---|---|---|---|---|---|
| Left-NIaIV | plus | 89 | 89 | 89 | 27632.512 | 27632.512 | 27632.512 |
| | minus | 89 | 89 | 89 | 23764.952 | 23764.952 | 23764.952 |
| NIaIV-Tsp509I | plus | 15 | 15 | 15 | 4694.775 | 4694.775 | 4694.775 |
| | minus | 19 | 19 | 19 | 5839.957 | 5839.957 | 5839.957 |
| Tsp509I-Tsp509 | plus | 18 | 18 | 18 | 5657.958 | 5657.958 | 5657.958 |
| | minus | 18 | 18 | 18 | 5492.881 | 5492.881 | 5492.881 |
| Tsp509I-NIaIV | plus | 40 | 40 | 40 | 12273.955 | 12273.955 | 12273.955 |
| | minus | 36 | 36 | 36 | 11227.901 | 11227.901 | 11227.901 |
| NIaIV-Right | plus | 118 | 115 | 115 | 36580.077 | 35667.940 | 35673.928 |
| | minus | 118 | 115 | 115 | 36311.913 | 35372.741 | 35365.758 |

CFTR exon 10 amplicon
wt is 280 bp
delta508 is 277 bp
delta507 is 277 bp

CFTR amplicons whose size is within the resolving range of FT-ICR are analyzed for mass variation without fragmentation. These amplicons will be examined for mass variation either individually or as mixtures with other amplicons that are also within the resolving range of the FT-ICR.

Amplicons whose size is beyond the resolving range of FT-ICR are fragmented prior to analysis for mass variation, as described in Example 3. Based on the frequency of occurrence of restriction enzyme sites within a designated amplicon, amplicons are digested using one or more restriction enzymes to cleave the DNA such that the resulting fragments are less than, e.g., about 100 bp in length. The amplicons are singly digested or, alternatively, mixed in different combinations such that mix 1, comprised of two or more amplicons, is digested with a combination of restriction enzymes, e.g. RE 1-3. Then, mix 2, also comprised of two or more amplicons, is digested with a combination of restriction enzymes, e.g. RE 1, 3, and 4. Additional amplicon mixes are assembled and digested appropriately to generate RE fragments whose sizes are within the range of resolution by mass spectrometry and can be unambiguously distinguished from other fragments within the digest by fragment mass determinations utilizing mass spectrotrometers (MS), preferably utilizing ESI-FTICR. Mass spectrometers such as these are able to determine M/Z with high range, resolution, and accuracy e.g. ≦200 bp, 30,000 and >0.01%, respectively.

To analyze Mendelian inheritance of genetic diseases or disease predispositions, it is beneficial to have access to genomic DNA from the parents, siblings, and other first-degree relatives in addition to the test subject (the proband). Accordingly, amplification of the exons and splice regions of the CFTR gene is performed for each member in the family for which genomic DNA is available (FIG. 8). Once amplified, each set of amplicons for individual family members are fragmented, analyzed by ESI-FTICR and then compared to a reference set of amplicons derived from genomic DNA of known sequence, or alternatively, compared to a database containing masses of predicted amplicons. Mass analyses that reveal differences between one or more amplicons (and resulting RE fragments) derived from test DNAs and the appropriate reference set of amplicons (and resulting RE fragments) will denote variant amplicons that encode a sequence different than that of the reference sequence. Furthermore, variant and invariant amplicons derived from the test subject (proband) should be consistent with Mendelian inheritance. Exceptions to this prediction may arise due to somatic mutations within the discordant amplicon. When mass variant amplicon mixes are identified, the mass analysis determination is repeated with individual amplicons that comprised the original amplicon mix to ascertain which amplicon or amplicons show mass variation. After identifying individual amplicons that fail to validate the reference sequence, those amplicons will be sequenced either completely or within intervals that will encompass restriction enzyme fragments of variant mass when compared to the standards predicted by the reference sequence.

### Equivalents

The invention now being fully described, it will be apparent to one of ordinary skill in the art that many changes and modifications can be made thereto without departing from the spirit or scope of the invention and the appended claims. Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents is selected for the present invention and embodiments thereof.

What is claimed is:

1. A method for validating the sequence of a test double stranded nucleic acid, said method comprising:
(a) contacting said test double stranded nucleic acid with one or more separation means, such that two or more double stranded nucleic acid fragments are generated from said test nucleic acid;
(b) generating one or more output signals from each of said double stranded nucleic acid fragments, said output signal comprising a representation of the molecular mass of each of said double stranded nucleic acid fragments; and
(c) comparing said one or more output signals with a set of output signals known or predicted to be produced by a double stranded reference nucleic acid of identical sequence to the predicted sequence of the test nucleic acid, whereby the sequence of said test nucleic acid is validated.

2. The method of claim 1, wherein said separation means is a recognition means.

3. The method of claim 2, wherein said recognition means is a restriction endonuclease.

4. The method of claim 3, wherein said restriction endonuclease is a type 2 restriction endonuclease.

5. The method of claim 1, wherein said generating one or more output signals comprises performing mass spectrometry on each of said fragments.

6. The method of claim 1, wherein mass spectrometry is selected from the group consisting of ion cyclotron resonance mass spectrometry, electrospray ionization fourier transform ion cyclotron resonance mass spectrometry, matrix-assisted laser desorption ionization mass spectrometry, quadropole ion trap mass spectrometry, magnetic/electric sector mass spectrometry and time-of-flight mass spectrometry.

7. The method of claim 1, wherein said target nucleic acid is DNA.

8. The method of claim 1, wherein said target nucleic acid is double stranded RNA.

9. The method of claim 1, further comprising repeating steps (a) and (b) one or more times.

10. The method of claim 1, further comprising repeating steps (a) and (b) one or more times, under conditions such that the size of each of the two or more nucleic acid fragments is decreased with each repetition.

11. The method of claim 1, wherein steps (a) and (b) are repeated three times, under conditions such that the size of each of the two or more nucleic acid fragments is decreased with each repetition.

12. The method of claim 3, wherein said two or more nucleic acid fragments are each under 500 bases in length.

13. The method of claim 3, wherein said two or more nucleic acid fragments are each under 200 bases in length.

14. The method of claim 3, wherein said two or more nucleic acid fragments are each under 100 bases in length.

15. The method of claim 3, wherein said two or more nucleic acid fragments are each under 75 bases in length.

16. The method of claim 3, wherein said two or more nucleic acid fragments are each under 50 bases in length.

17. The method of claim 3, wherein said two or more nucleic acid fragments are each under 20 bases in length.

18. A method for identifying a polymorphism in a test double stranded nucleic acid, said method comprising:
(a) contacting said test double stranded nucleic acid with one or more separation means, such that two or more double stranded nucleic acid fragments are generated from said test nucleic acid;
(b) generating one or more output signals from each of said fragments, said output signal comprising a representation of the molecular mass of each of said fragments; and
(c) comparing said one or more output signals with a set of output signals of a reference nucleic acid of identical sequence, whereby a difference in said one or more output signals of one or more nucleic acid fragments indicates a difference in the sequence of said one or more nucleic acid fragments, thereby identifying a polymorphism in said test nucleic acid.

19. The method of claim 18, further comprising:
(d) identifying said one or more nucleic acid fragments having said polymorphism; and
(e) repeating steps (a) through (c) one or more times, under conditions such that the size of each of the two or more nucleic acid fragments is decreased with each repetition.

20. The method of claim 18, further comprising:
(d) sequencing the nucleic acid fragments with output signals different from the output signals of the reference nucleic acid.

21. The method of claim 20, wherein the sequencing of nucleic acid fragments comprises a method chosen from the group consisting of Sanger sequencing, Maxam-Gilbert sequencing, pyro-sequencing, and sequencing by hybridization.

22. A method for detecting a polymorphism in a target nucleic acid, said method comprising obtaining from said target nucleic acid a population of nucleic acid fragments in double stranded form, wherein said population essentially comprises the entirety of fragments generated from non-randomly fragmenting a double-stranded target nucleic acid, and determining the molecular masses of each of the double-stranded nucleic acid fragments of said population.

23. The method of claim 22, further comprising comparing said molecular mass of each of the double-stranded nucleic acid fragments with the molecular masses known or predicted to be produced by a double stranded reference nucleic acid; and sequencing the nucleic acid fragments with molecular masses different from the molecular masses of the reference nucleic acid.

24. A method for detecting a variation in a nucleic acid sequence among two individuals, said method comprising:
(a) independently contacting a first nucleic acid from a first individual and a second nucleic acid from a second individual with one or more separation means, such that two or more double stranded nucleic acid fragments are generated from each of said first nucleic acid and said second nucleic acid;
(b) generating one or more output signals from each of said fragments, said output signal comprising a representation of the molecular mass of each of said fragments; and
(c) comparing said one or more output signals generated in step (b) from said first nucleic acid with said one or more output signals generated in step (b) from said second nucleic acid, whereby a variation in a nucleic acid sequence among two individuals is detected.

25. A method for determining paternity of an offspring, said method comprising:
(a) independently contacting a first nucleic acid from a first individual and a second nucleic acid from a second individual with one or more separation means, such that two or more double stranded nucleic acid fragments are generated from each of said first nucleic acid and said second nucleic acid;
(b) generating one or more output signals from each of said fragments, said output signal comprising a representation of the molecular mass of each of said fragments; and
(c) comparing said one or more output signals generated in step (b) from said first nucleic acid with said one or more output signals generated in step (b) from said second nucleic acid, thereby determining the paternity of said first individual relative to said second individual.

26. A method for identifying a polymorphism in a target double stranded nucleic acid, said method comprising:
(a) contacting said target double stranded nucleic acid with one or more restriction enzymes, such that two or more double stranded nucleic acid fragments are generated from said target nucleic acid;
(b) determining the molecular masses of each of the double-stranded nucleic acid fragments;
(c) comparing the molecular masses of each of the double-stranded nucleic acid fragments with the molecular masses of the double-stranded nucleic acid fragments known or predicted to be produced by a double stranded reference nucleic acid of identical sequence to the target nucleic acid;
(d) repeating steps (a) through (c) three times, under conditions such that the size of each of the two or more nucleic acid fragments is decreased with each repetition; and
(e) sequencing the nucleic acid fragment(s) with molecular masses different from the molecular masses of the double-stranded nucleic acid fragments of the reference nucleic acid.

27. A method for analyzing a target double stranded nucleic acid, said method comprising:
(a) amplifying two or more nucleic acid subsequences from said target nucleic acid;
(b) determining the molecular masses of each of the amplified nucleic acid subsequences;
(c) comparing the molecular masses of each of the amplified nucleic acid subsequences with the molecular masses of the amplified nucleic acid subsequences known or predicted to be produced by amplification of a double stranded reference nucleic acid of identical sequence to the target nucleic acid,
thereby analyzing the target double stranded nucleic acid.

28. The method of claim 27, further comprising digesting said amplified nucleic acid subsequences with one or more restriction endonucleases prior to determining the molecular masses of each of the amplified nucleic acid subsequences.

29. The method of claim 27, wherein said target double stranded nucleic acid is genomic DNA.

30. The method of claim 27, wherein a portion of each of said amplified nucleic acid subsequences overlaps a portion of at least one other amplified nucleic acid subsequence.

31. The method of claim 27, wherein no portion of each of said amplified nucleic acid subsequences overlaps with any portion of any other amplified nucleic acid subsequence.

32. A processor for analyzing nucleic acid sequences comprising:
a selecting module that enables a user to select one or more textual strings corresponding to one or more genes;
in response to the user's selection, a providing module that provides a first set of nucleic acid sequence fragments comprising the fragments predicted to be generated by contacting a first double stranded nucleic acid molecule with at least one separation means, said first set of nucleic acid sequence fragments associated with the selected one or more textual stings;
an evaluating module that evaluates each of the first set of nucleic acid sequence fragments to predict the mass of each fragment of the first set of nucleic acid sequence fragments;
a retrieving module that retrieves experimental results comprising the mass of each of a second set of nucleic acid sequence fragments, said second set of nucleic acid sequence fragments generated by contacting a second double stranded nucleic acid molecule with said at least one separation means;
a validating module that validates each of the first set of nucleic acid sequence fragments by evaluating the mass of each fragment of the first set of nucleic acid sequence fragments against the mass of each fragment of the second set of nucleic acid sequence fragments.

33. The processor of claim 32 further comprising a storing module that stores the results of the validation.

34. The processor of claim 32, wherein said separation means is a recognition means.

35. The processor of claim 33, wherein said recognition means is a restriction endonuclease.

36. The processor of claim 35, wherein said restriction endonuclease is a type 2 restriction endonuclease.

37. The processor of claim 32, wherein said evaluating the mass of each fragment comprises performing mass spectrometry on each fragments.

38. The processor of claim 37, wherein mass spectrometry is selected from the group consisting of ion cyclotron resonance mass spectrometry, electrospray ionization fourier transform ion cyclotron resonance mass spectrometry, matrix-assisted laser desorption ionization mass spectrometry, quadropole ion trap mass spectrometry, magnetic/electric sector mass spectrometry and time-of-flight mass spectrometry.

39. The processor of claim 32, wherein said nucleic acid is DNA.

40. The processor of claim 32, wherein said nucleic acid is double stranded RNA.

41. A method for analyzing nucleic acid sequences comprising:
enabling a user to select one or more textual strings corresponding to one or more genes;
in response to the user's selection, providing a first set of nucleic acid sequence fragments associated with the selected one or more textual strings, said first set of nucleic acid sequence fragments comprising the fragments predicted to be generated by contacting a first double stranded nucleic acid molecule with at least one separation means;
evaluating each of the first set of nucleic acid sequence fragments to predict the mass of each of the first set of nucleic acid sequence fragments;
retrieving experimental results comprising the mass of each of a second set of nucleic acid sequence fragments, said second set of nucleic acid sequence fragments generated by contacting a second double stranded nucleic acid molecule with said at least one separation means; and
validating the each of the first set of nucleic acid sequence fragments by evaluating the mass of the each of the first set of nucleic acid sequence fragments against the mass of each of the second set of nucleic acid sequence fragments.

42. The method of claim 41 further comprising storing the results of the validation.

43. The method of claim 41, wherein said separation means is a recognition means.

44. The method of claim 41, wherein said recognition means is a restriction endonuclease.

45. The method of claim 44, wherein said restriction endonuclease is a type 2 restriction endonuclease.

46. The method of claim 41, wherein said evaluating the mass of each fragment comprises performing mass spectrometry on each fragments.

47. The method of claim 46, wherein mass spectrometry is selected from the group consisting of ion cyclotron resonance mass spectrometry, electrospray ionization fourier transform ion cyclotron resonance mass spectrometry, matrix-assisted laser desorption ionization mass spectrometry, quadropole ion trap mass spectrometry, magnetic/electric sector mass spectrometry and time-of-flight mass spectrometry.

48. The method of claim 41, wherein said nucleic acid is DNA.

49. The method of claim 41, wherein said nucleic acid is double stranded RNA.

50. A processor for analyzing nucleic acid sequences comprising:
selecting means that enables a user to select one or more textual strings corresponding to one more genes;
in response to the user's selection, providing means that provides the mass of each fragment of a first set of nucleic acid sequence fragments associated with the selected one or more textual strings;
evaluating means that evaluates each of the first set of nucleic acid sequence fragments to predict the mass of each fragment of the first set of nucleic acid sequence fragments for at least one separation means;
retrieving means that retrieves experimental results comprising the mass of each fragments in a second set of nucleic acid sequence fragments for said at least one separation means;
validating means that validates the first set of nucleic acid sequence fragments by evaluating the mass of each fragment of the first set of nucleic acid sequence fragments against the experimental results of the mass of each fragment of the second set of nucleic acid sequence fragments; and
storing means that stores the results of the validation.

51. A processor readable medium for analyzing nucleic acid sequences, said medium comprising:
a first processor readable program code for enabling a user to select one or more textual strings corresponding to one or more genes;
in response to the user's selection, a second processor readable program code for providing a first set of nucleic acid sequence fragments associated with the selected one or more textual strings;
a third processor readable program code for evaluating each of the first set of nucleic acid sequence fragments to calculate the mass of each fragment of the first set of nucleic acid sequence fragments, said first set of nucleic acid sequence fragments comprising the fragments predicted to be generated by contacting a first double stranded nucleic acid molecule with at least one separation means;
a fourth processor readable program code for retrieving experimental results of the determination of the mass of each fragment of a second set of nucleic acid sequence fragments, said second set of nucleic acid sequence fragments comprising the fragments generated by contacting a second double stranded nucleic acid molecule with said at least one separation means;
a fifth processor readable program code for validating the sequence of the first nucleic acid molecule by evaluating the mass of each fragment of the first set of nucleic acid sequence fragments against the experimental results of the mass of each of the second set of nucleic acid sequence fragments; and
a sixth processor readable program code for storing the results of the validation.


SEQUENCE LISTING (4 sequences)

SEQ ID NO: 1 - DNA, 1944 bp, Mesocricetus auratus
atgatgaaccagtctcagagaatggcacctgtgggctccgacaaagagctgagtgatctcctggacttcagtatgatgttcccgctccctgtggccaacgggaagggccggcccgcctccctagctggaacgcagtttgcaggctcaggacttgaggaccgacccagctcaggctcctggggcaacagtgatcagaacagctcttccttcgaccccagcaggacgtacagcgagggcgcccactttagcgagtcccacaacagcctgccttcttccacgttcttaggacctgggcttggaggcaaaagcagcgagcggagtgcttatgccaccttcgggagagacaccagtgttagtgcactgactcaggctggcttcctgccgggtgagctgggccttagtagccctgggccactgtctccatcgggtgtcaagagcggctcccagtattatccctcataccccagcaaccctcggcggagagctgcagacagtggcctggatacacagtccaagaaggtccggaaggttccacctggtctgccctcctctgtgtatccgtccagctcaggtgacagctacggcagggatgccgcggcctacccctctgccaagacccctggcagtgcctatccctcccctttctacgtggcagatggcagcctgcacccctctgcggagctttggagtccccccagccaggcgggctttgggcccatgttaggtgacggctcgtcccctctgccccttgccccaggcagcagttccgtgggcagtggcacctttgggggtctccagcagcaggaacgcatgagctaccagctgcacgggtctgaggtcaacggcacgctcccagctgtgtccagcttctcagccgcccctggcacttatggtggggcttctggtcacacaccacctgtgagcggggccgacagcctcatgggcacccgagggactacagccagcagctcaggggatgcccttgggaaggcgctggcctcgatctactccccggatcactccagcaataacttctcacccagcccctcgacgcctgtgggttcaccccagggcctgccagggacatcacagtggccccgggcaggagcgcccagtgccttatctcccacctacgacgggggtctccatggcctgcagagcaagatggaggatcgcttggatgaggccatccatgtccttcgaagccacgctgtgggcaccgctagcgatctccatggacttctgcctggccatggggcactgaccactagcttccctggccccgtgccactgggcgggcggcatgcgggcctggttggtggcggccaccctgaggatggcctcaccagtggcactagtcttttgcatacccatgccagcctccccagccaggccagctccctccccgacctctcgcagaggccaccggactcttacggcggactaggaagggcaggtgccccagccggcgccagcgagatcaagcgggaggagaaagacgacgaggagagcacctcagtggccgacgccgaggaggacaagaaggacctgaaggctccacgcacgcgcaccagcagtacggacgaggtgctgtccctggaggagaaggacctgagggaccgggagaggcgcatggccaataacgcccgggagcgggtgcgcgtgcgggacattaacgaggccttccgggagctgggccgcatctgccagctgcacctcaagtcggataaggcgcagaccaagctgctgatcctgcagcaggcggtgcaggttatcctgggcctggagcagcaggtgcgagagcgcaacctgaaccccaaagcagcctgcttgaagcggagggaggaggagaaggtgtctggcgtggtcggggacccccagctggcgctgtctgctgcccaccctggcctgggtgaggcccacaacccgcccgggcacctgtga

SEQ ID NO: 2 - DNA, 1950 bp, Mesocricetus auratus
atgatgaaccagtctcagagaatggcacctgtgggctccgacaaagagctgagtgatctcctggacttcagtatgatgttcccgctccctgtggccaacgggaagggccggcccgcctccctagctggaacgcagtttgcaggctcaggacttgaggaccgacccagctcaggctcctggggcaacagtgatcagaacagctcttccttcgaccccagcaggacgtacagcgagggcgcccactttagcgagtcccacaacagcctgccttcttccacgttcttaggacctgggcttggaggcaaaagcagcgagcggagtgcttatgccaccttcgggagagacaccagtgttagtgcactgactcaggctggcttcctgccgggtgagctgggccttagtagccctgggccactgtctccatcgggtgtcaagagcggctcccagtattatccctcataccccagcaaccctcggcggagagctgcagacagtggcctggatacacagtccaagaaggtccggaaggttccacctggtctgccctcctctgtgtatccgtccagctcaggtgacagctacggcagggatgccgcggcctacccctctgccaagacccctggcagtgcctatccctcccctttctacgtggcagatggcagcctgcacccctctgcggagctttggagtccccccagccaggcgggctttgggcccatgttaggtgacggctcgtcccctctgccccttgccccaggcagcagttccgtgggcagtggcacctttgggggtctccagcagcaggaacgcatgagctaccagctgcacgggtctgaggtcaacggcacgctcccagctgtgtccagcttctcagccgcccctggcacttatggtggggcttctggtcacacaccacctgtgagcggggccgacagcctcatgggcacccgagggactacagccagcagctcaggggatgcccttgggaaggcgctggcctcgatctactccccggatcactccagcaataacttctcacccagcccctcgacgcctgtgggttcaccccagggcctgccagggacatcacagtggccccgggcaggagcgcccagtgccttatctcccacctacgacgggggtctccatggcctgagcaagatggaggatcgcttggatgaggccatccatgtccttcgaagccacgctgtgggcaccgctagcgatctccatggacttctgcctggccatggggcactgaccactagcttccctggccccgtgccactgggcgggcggcatgcgggcctggttggtggcggccaccctgaggatggcctcaccagtggcactagtcttttgcatacccatgccagcctccccagccaggccagctccctccccgacctctcgcagaggccaccggactcttacggcggactaggaagggcaggtgccccagccggcgccagcgagatcaagcgggaggagaaagacgacgaggagagcacctcagtggccgacgccgaggaggacaagaaggacctgaaggctccacgcacgcgcaccagcccagacgaggacgaggacgaccttctccccccagagcagaaggccgagcgggagaaggagcgccgggtggccaataacgcccgtgagcgcctgcgggtccgcgacatcaatgaggcctttaaggagctgggccgcatgtgccagctgcacctcagcagtgagaagccgcagaccaaactgctcatcctgcaccaggccgtggccgtcatcctcagcctggagcagcaggtgcgagagcgcaacctgaaccccaaagcagcctgcttgaagcggagggaggaggagaaggtgtctggcgtggtcggggacccccagctggcgctgtctgctgcccaccctggcctgggtgaggcccacaacccgcccgggcacctgtga

SEQ ID NO: 3 - DNA, 11 bp, Artificial Sequence
  Description of Artificial Sequenceconsensus sequence
gccnnnnnggc

SEQ ID NO: 4 - DNA, 12 bp, Artificial Sequence
  Description of Artificial Sequenceconsensus sequence
cgannnnnntgc